(19) United States
(12) Patent Application Publication
(10) Pub. No.: US 2005/0089923 A9
(43) Pub. Date: Apr. 28, 2005

(54) Method and system for planning, performing, and assessing high-throughput screening of multicomponent chemical compositions and solid forms of compounds

(76) Inventors: Douglas A. Levinson, Sherborn, MA (US); Christopher McNulty, Minneapolis, MN (US); Christopher B. Moore, Cambridge, MA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/142,812

(22) Filed: May 10, 2002

Related U.S. Application Data
(63) Continuation-in-part of application No. 10/103,983, filed on Mar. 22, 2002.
Application No. 10/103,983 is a continuation-in-part of application No. 09/756,092, filed on Jan. 8, 2001.
Application No. 10/103,983 is a continuation-in-part of application No. 09/994,585, filed on Nov. 27, 2001.
Application No. 09/994,585 is a continuation-in-part of application No. 09/756,092, filed on Jan. 8, 2001.
Application No. 09/994,585 is a continuation-in-part of application No. PCT/US01/00531, filed on Jan. 8, 2001.
Application No. 10/103,983 is a continuation-in-part of application No. PCT/US01/44818, filed on Nov. 28, 2001.
(63) Continuation-in-part of application No. 09/756,092, filed on Jan. 8, 2001.
(63) Continuation-in-part of application No. 09/994,585, filed on Nov. 27, 2001.
(63) Continuation-in-part of application No. PCT/US01/44818, filed on Nov. 28, 2001.
(60) Provisional application No. 60/290,320, filed on May 11, 2001.
(60) Provisional application No. 60/221,539, filed on Jul. 28, 2000.
(60) Provisional application No. 60/196,821, filed on Apr. 13, 2000.
(60) Provisional application No. 60/175,047, filed on Jan. 7, 2000.
(60) Provisional application No. 60/175,047, filed on Jan. 7, 2000.
(60) Provisional application No. 60/196,821, filed on Apr. 13, 2000.
(60) Provisional application No. 60/221,539, filed on Jul. 28, 2000.
(60) Provisional application No. 60/253,629, filed on Nov. 28, 2000.
(60) Provisional application No. 60/253,629, filed on Nov. 28, 2000.

(51) Int. Cl. G01N 33/53; G06F 19/00; G01N 33/48; G01N 33/50

(52) U.S. Cl. 435/7.1; 702/19

(57) ABSTRACT

A method and system for planning and assessing the results of high-throughput solid form screening and high-throughput formulation screening are disclosed. Also disclosed are methods and systems for using high-throughput solid form screening and high-throughput formulation screening to select compounds and formulations for further testing, or to prioritize testing.

This application claims the benefit of U.S. Application No. 60/290,320 entitled METHOD AND SYSTEM FOR PLANNING, PERFORMING, AND ASSESSING HIGH-THROUGHPUT SCREENING OF MULTICOMPONENT CHEMICAL COMPOSITIONS AND SOLID FORMS OF COMPOUNDS, filed on May 11, 2002, which is incorporated herein in its entirety by reference.

### FIELD OF THE INVENTION

The present invention relates to the field of computerized data processing of experimental data relating to chemical compounds or compositions and formulations and solid forms of chemical compounds or compositions.

### BACKGROUND OF THE INVENTION

Most chemical products embody compromises. In pharmaceuticals, for example, there are typically trade-offs between drug solubility, stability, absorption and bioavailability. Flubxetine, the active agent in PROZAC®, suffers from very low solubility in water and undergoes extensive first pass hepatic metabolism. Loratadine, the active agent in CLARITIN®, is insoluble in water and also undergoes extensive first pass metabolism in the liver. The active agent in TAXOL®, paclitaxel, suffers from poor absorption due to its low water solubility.

In some cases these trade-offs can be usefully manipulated through changes of the solid form and/or the chemical formulation in which an active agent is delivered. The solubility, bioavailability, shelf-life, usability, taste and many other properties of the chemical product may vary in a complex way with the formulation due to interactions among the active agent and the excipients that make up the chemical product, and the particular use or administration method, thereof. Similarly, properties of the solid form of an ingredient, such as its crystal habit and morphology, can significantly affect properties such as stability, bioavailability, and industrial processing. Selection of optimal formulations and solid form can therefore significantly alter the performance of pharmaceuticals and other chemical products. Dietary supplements, alternative medicines, nutraceuticals, sensory compounds, agrochemicals, and consumer and industrial formulations, also can benefit from reformulation and new solid forms.

Failure to explore alternative formulations and solid forms may result not only in the marketing of a sub-optimal form or formulation, but can even result in failure of a product or formulation that is chosen without knowledge of the variety of solid forms the active ingredient may take, or the behavior of the chosen form or formulation over a range of conditions likely to be encountered in manufacturing or the marketplace. A commercially significant example of a pharmaceutical which suffered from difficulties in formulation and manufacturing due to a crystal polymorph unknown at the time the drug was initially marketed for sale is Ritonavir. Ritonavir is a protease inhibitor used to treat human immunodeficiency virus infection marketed by Abbot Laboratories as NORVIR. NORVIR brand ritonavir was introduced in 1996 as a semisolid capsule formulation and as a liquid formulation. In 1998, many lots of the Norvir capsules started to fail dissolution testing, because a large portion of the active pharmaceutical ingredient (ritonavir) was precipitating out of the semisolid formulated product. It was discovered that a previously unknown crystal polymorph, called Form II, was in the precipitates. Form II is more thermodynamically stable than Form I, and has a much lower solubility in the solvents used to formulate the NORVIR product, so that the formulation was very supersaturated with respect to Form II.

Form II continued to be produced and precipitate out during the manufacturing process to the point where all attempts to formulate the semisolid capsules were unsuccessful, and this quickly caused a shortage of the product and result in a marketing crisis for Abbott. In addition, while attempting to address the problem, Abbott encountered the further problem that their methods for synthesizing ritonavir, both at the bench level an in Abbott's bulk drug manufacturing process, now could not even synthesize Form I ritonavir, either at the bench scale or in bulk drug manufacturing processes, as all attempts to synthesis Form I resulted in production of Form II. Chemburkar, et al., “Dealing with the Impact of Ritonavir Polymorphs on the Late Stages of Bulk Drug Process Development,” Organic Process Res. Dev., 4:413-417 (2000).

Given the large resources that are required at each stage of the drug research and development process, and the high percentage of candidate compounds that fail to make it through the research and development process, there is a need to determine at each stage of the process whether a candidate compound may be manufactured in an appropriate solid form and suitably formulated.

Traditionally, attempts to identify what solid-forms of a compound exist has been a tedious, labor-intensive, and time-consuming process that generally focuses on finding only an apparently suitable solid-form without exploring further to determine whether other solid-forms also exist. Furthermore, scaling up synthesis methods or manufacturing processes often introduces process variables or conditions that are more difficult or expensive to control than in the laboratory, and crystallization conditions that work well on an experimental scale may not work well on larger (e.g., industrial) scales. As a result, unexpected process, manufacturing or formulation problems can developed at various stages of the research and development processes, sometimes as late as after product launch as seen in the NORVIR situation. A need therefore exists for a rapid and systematic method of identifying various possible solid-forms of a compound-of-interest, and the ranges of conditions which may be used in manufacturing processes to consistently and economically produce the desired solid form.

A need also exists for a rapid and systematic process to identify methods of manufacture or making a desired solid-form that are not susceptible to the potential effects of process impurities or degradants. Process impurities and degradant that are formed during the manufacture of a particular compound can profoundly impact the crystallization of that compound, e.g., by inhibiting nucleation or crystal growth. Such process impurities or degradants may resemble the compound-of-interest, and be selectively absorbed by a crystal nucleus of the compound, possibly functioning as a potent growth inhibitor. Inhibition of a desired polymorph may result in nucleation and growth of an undesired polymorph, as thought to be the case for ritonavir. Bauer et al., Pharm. Res. 18:859-866 (2001).

Similarly, the task of determining an optimal or near-optimal formulation is enormous. On the one hand, a property often can be optimized only at the expense of other desirable properties, so that no single property may be optimized in isolation. On the other, the properties of compounds or mixtures vary in a complex or unpredictable way with formulation parameters. Also, the types and ranges of formulation parameters that may be varied in manufacturing are very large.

For example, more than 3,000 excipients are currently available for designing pharmaceutical compositions. A search for an optimum combination of excipients and active agents for even a relatively simple pharmaceutical composition has been unfeasible in the past. Not only does one need to determine which of those excipients would be compatible with the active agent, but one has to determine the optimum values for such parameters as pH and relative concentrations of the components. As a result, conventional formulation techniques have generally been a search for an adequate formulation in an adequate time period, rather than a search for an optimum or near-optimum among significant numbers of adequate formulations. Indeed, to avoid a difficult search for an adequate formulation, new active agents are often “force fitted” into standard formulation recipes that are modified as little as possible to result in a adequate formulation.

The problem grows geometrically with the number of excipients and other parameters considered. For example, simply to select a combination of two excipients out of a group of three hundred, without considering other variables such as relative concentrations, requires sifting through 44,850 combinations. This increases rapidly to 4,455,100 combinations for three compounds, and 330,791,175 combinations in the case of a four-compound mixture. Similar problems confront an effort to develop new solid forms of known substances.

In addition, because the conditions under which a formulation or solid form is manufactured, stored, administered or used typically vary over a significant range, the commercial usefulness of a formulation or solid form depend on the properties of the formulation or solid form over the expected range of conditions under which it will be manufactured, stored, administered or used. If the properties of the formulation change significantly over the expected range, or if the solid form is unstable or another solid form is produced at different points of the expected range, the usefulness of the formulation or solid form can decrease. Selection of a commercially-useful formulation or solid form therefore benefits from consideration of the behavior of the formulation or solid form over the expected range.

The scale of these problems may be reduced if relationships between one or more properties to be optimized and one or more molecular descriptors are discovered. A molecular descriptor as used herein is an empirical or theoretical datum that may be used in a quantitative structure-activity or structure-property relationship to predict molecular properties in complex environments. For a discussion of molecular descriptors, see Karelson, Molecular Descriptors in QSAR/QSPR, John Wiley & Sons, Inc. (2000), which is incorporated herein by reference. Many categories of compounds, such as pharmaceutical excipients, have been characterized based on a large number of molecular descriptors. Commercial and noncommercial databases of such characterizations are often available. Typically the molecular descriptors relevant to a desired property or properties are a small fraction of those that are measurable, calculable, or known. Moreover, the relationship between the relevant molecular descriptors and the desired property or properties often cannot be easily determined.

The magnitude of the problem does not arise solely from the extremely large number of possible combinations of relevant parameters that may be varied in manufacturing or experimentation. In many situations, neither the experimentally variable parameters nor the measurable or calculable characteristics of a compound or mixture of interest will have any known correlation with the property or properties which the experimentalist seeks to optimize. In the past, attempts have been made to characterize a material by performing one experiment at a time using a preselected combination of molecular descriptors and/or one or more bulk properties. This method of characterization is very time-consuming.

Recent advances in automation of experiments or experimental procedures have made it possible to perform tens or even hundreds of thousands of experiments in a relatively short period. Nevertheless, because the number and range of experimental parameters available to the experimentalist are extremely large, even hundreds of thousands of data points may be a very small fraction of accessible experiments that may be relevant to the properties of interest. Also, because the measured results may vary in a highly non-linear fashion with the experimental parameters, unsophisticated selection of even a large number of data points may not accurately characterize the relationship between measured properties and experimental parameters. Thus, one may be able to collect hundreds of thousands of experimental data points and still fail to determine useful correlations or relationships between experimental or manufacturing parameters and desired properties. The range of possible experiments is likely to be too large for random or uniform sampling alone to yield optimal or near-optimal results.

There is thus a need to systematically integrate all available information in a manner that permits the useful deployment of a limited number of experiments to assess with confidence the commercial potential of a compound-of-interest (including various solid-forms and formulations thereof), and to increase or maximize the probability of yielding compounds, compositions, or formulations that possess a desired property or set of properties over an expected range of conditions of manufacture, storage, administration or use, or combinations thereof.

### SUMMARY OF THE INVENTION

In one aspect, the present invention comprises a method for determining a formulation of a pharmaceutical, comprising the steps of: performing high-throughput formulation screening of the pharmaceutical; computing an optimization algorithm to select a plurality of molecular descriptors and a model accepting the molecular descriptors as parameters to optimize the predictive power of the model; determining the formulation of the pharmaceutical.

In a related aspect, the present invention comprises a method for generating a plurality of solid forms of a pharmaceutical, comprising the steps of: performing high-throughput solid-form screening of the pharmaceutical; computing an optimization algorithm to select a plurality of molecular descriptors and a model accepting the molecular descriptors as parameters to optimize the predictive power of the model; determining the formulation of the pharmaceutical.

In another aspect the foregoing methods may further comprise the steps of: generating values of experimental parameters using the model; performing high-throughput screening using the generated values. comparing the high-throughput experimental results with the results predicted by the model; adjusting the model based on the high-throughput experimental results.

In the foregoing methods, the generated values are preferably targeted to find an extremum of an expected property of an experiment, boundaries between solid forms, regions in which desired properties of formulations change rapidly with respect to changes experimental parameters, regions in which desired properties of formulations change slowly with respect to changes experimental parameters or regions of ambiguity or low confidence in classification or regression results.

Preferably, the predictive power is determined with respect to an extremum of an expected property of an experiment, with respect to boundaries between solid forms, with respect to regions in which desired properties of formulations or solid forms change rapidly with respect to changes in experimental parameters, or with respect to one or more regions within class boundaries.

A variety of optimization algorithms and models may be used. In one embodiment, an approximately maximally diverse set of values of experimental parameters for high-throughput screening is generated using a diversification algorithm and a metric for measuring diversification. In another embodiment, a set of values of experimental parameters for high-throughput screening is generated based on a structure-activity model.

In another aspect, the present invention comprises a method for selecting a compound for further testing, comprising the steps of: receiving information of a plurality of compounds; performing high-throughput solid-form screening of at least one of the plurality of compounds to identify at least one solid-form; based on the at least one property of each identified solid-form, selecting at least one of the plurality of compounds for further testing.

In a related aspect, the present invention comprises a method for selecting a compound for further testing, comprising the steps of: receiving information of a plurality of compounds; performing high-throughput formulation screening on at least one of the plurality of compounds; based on at least one tested property, selecting at least one of the plurality of compounds for further testing.

In another aspect, the present invention comprises a method for selecting a solid form of a compound for further testing, comprising the steps of: receiving information of a compound; performing high-throughput solid-form screening to identify at least two solid forms of the compound; based on the results of the high-throughput solid-form screening, selecting a solid form of the compound for further testing.

In still another aspect, the present invention comprises a method for selecting a formulation of a compound for further testing, comprising the steps of: receiving information of a compound; performing high-throughput formulation screening of the compound; based on the results of the high-throughput formulation screening, selecting a formulation of the compound for further testing.

In yet another aspect, the present invention comprises a method for determining whether to further test at least one compound, comprising the steps of: receiving information of the at least one compound; performing high-throughput formulation screening of the at least one compound; based on at least one tested property, determining whether to further test the at least one compound.

In another related aspect, the invention comprises a method for determining whether to further test at least one compound, comprising the steps of: receiving information of the at least one compound; performing high-throughput solid-form screening of the at least one compound; based on at least one tested property, determining whether to further test the at least one compound.

The foregoing methods may preferably further comprise the step of: based on the results of the high-throughput screening, generating a model to estimate at least one property of the compound. A variety of models may be used, and the methods are applicable to a variety of properties described below.

In one preferred embodiment, the methods of the present invention further comprise the steps of: based on the results of the high-throughput screening, generating a classifier to assign each solid form to a class. The classes may correspond to a variety of solid forms of a compound. In another embodiment, the methods further comprise the step of applying at least one unsupervised learning or clustering algorithm to at least a subset of the results of the high-throughput screening. A variety of unsupervised or clustering algorithms may be used as described below.

In another aspect, the methods may be used to prioritize testing. They may also be used to select a solid form based on high-throughput formulation testing. These and other embodiments are described below.

### BRIEF DESCRIPTION OF THE DRAWINGS

FIG. 1 is a schematic illustration of one example preferred embodiment.

FIG. 2 is an illustration of a display of a high-dimensional visualization in which the experimental results are represented as points of varying size, in a representation of a projection of a multidimensional space.

FIG. 3 is an illustration of an identification of certain groups of experimental results as exhibiting measured results of interest.

FIG. 4 is an illustration of additional data points corresponding to distinct experiments to characterize a formulation at higher resolution near results of interest.

FIG. 5 schematically illustrates a preferred method to assess a first collection of experimental results in a search for novel or known solid forms.

FIG. 6 schematically illustrates an architecture of a preferred example embodiment.

FIG. 7 depicts an example multivariate display.

FIG. 8 schematically depicts a preferred method to plan and assess experiments.

FIG. 9 schematically depicts a preferred method to plan and asses experiments.

FIG. 10 schematically depicts stages of clustering spectra.

FIG. 11 depicts an example filtered and unfiltered Raman spectrum.

FIG. 12 depicts a multivariate display of a dendrogram-sorted Tanimoto matrix.

FIG. 13 schematically depicts a simplified set of stages of pharmaceutical compound development and a corresponding qualitative indication of the reduction in the number of compounds at each stage in the process.

FIG. 14 schematically depicts a simplified overview of the pharmaceutical research and development process.

FIG. 15 schematically depicts a simplified overview of the pharmaceutical research and development process.

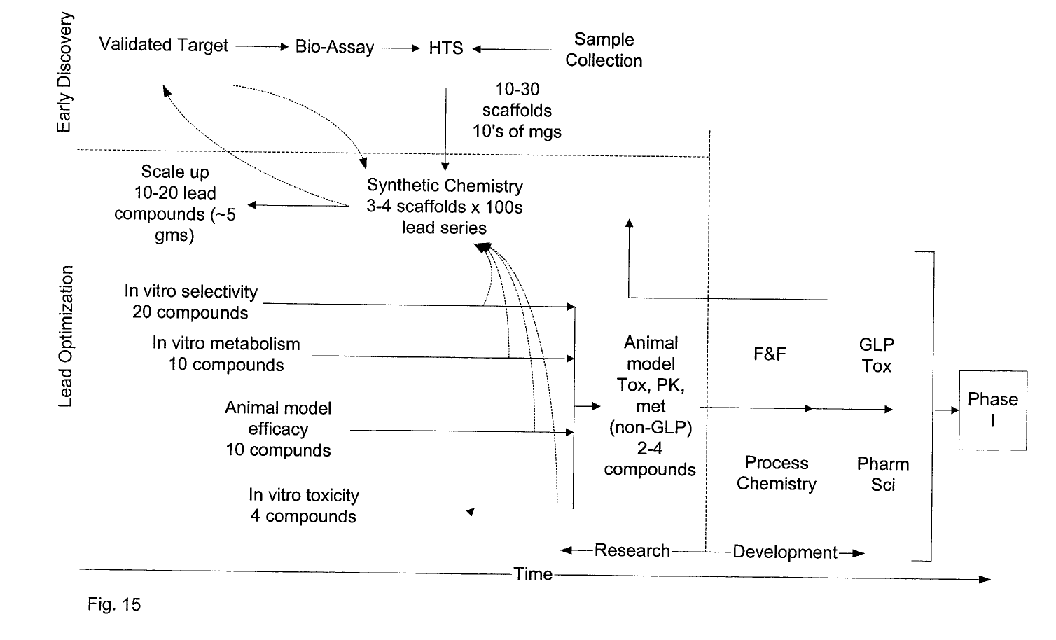

### DETAILED DESCRIPTION OF THE INVENTION

The present invention provides a system and associated methods for chemical knowledge acquisition through data acquisition, retrieval, and mining technologies, methods for applying the system and associated methods to assess whether a compound has properties suitable for commercial use, and for directing research and development expenditures towards compounds more likely to prove suitable for commercial uses, and away from compounds having properties that make commercial uses more difficult or impossible. Substances, such as pharmaceutical compounds can assume many different crystal forms and sizes. Particular emphasis has been put on these crystal characteristics in the pharmaceutical industry—especially polymorphic form, crystal size, crystal habit, and crystal-size distribution.—since crystal structure and size can affect manufacturing, formulation, and pharmacokinetics, including bioavailability. There are four broad classes by which crystals of a given compound may differ: composition, habit, polymorphic form, and crystal size.

As used herein, composition refers to whether the solid-form is a single compound or a mixture of compounds. For example, solid-forms can be present in their neutral form, e.g., the free base of a compound having a basic nitrogen or as a salt, e.g., the hydrochloride salt of a basic nitrogen-containing compound. Composition also refers to crystals containing adduct molecules. During crystallization or precipitation an adduct molecule (e.g., a solvent or water) can be incorporated into the matrix, adsorbed on the surface, or trapped within the particle or crystal. Examples include hydrates (water molecule incorporated in the matrix) and solvates (solvent trapped within a matrix). Whether a crystal forms as a hydrate or solvate can have a profound effect on the properties, such as the bioavailability or ease of processing or manufacture of a pharmaceutical. For example, hydrates or solvates may dissolve more or less readily, have different mechanical properties or strength, or have different physical and/or chemical stability than the corresponding non-hydrated or -solvated compounds.

A crystal habit refers to the external shape that a crystal assumes upon crystallization, which depend on, among others, the composition of the crystallizing medium. Those shapes may be cubic, tetragonal, orthorhombic, monoclinic, triclinic, rhomboidal, or hexagonal. Such information is important because the crystal habit has a large influence on the crystal□s surface-to-volume ratio. Although a single crystal polymorph may have different crystal habits, each having the same internal structure and thus the same single crystal- and powder-diffraction patterns, different crystal habits can exhibit different pharmaceutical properties (Haleblian 1975, J. Pharm. Sci., 64:1269). Thus discovering conditions or excipients that affect crystal habit are needed.

Polymorphism refers to the phenomenon in which a compound crystallizes into more than one distinct crystalline species (i.e., having a different internal structure) or shift from one crystalline species to another. The distinct species, which are known as polymorphs, can exhibit different optical properties, melting points, solubilities, chemical reactivities, physical stability, dissolution rates, and different bioavailabilities. It is well known that different polymorphs of the same pharmaceutical can have different pharmacokinetics, for example, different polymorphs may give rise to different levels of absorpotion of the compound. In the extreme, only one polymorphic form of a given pharmaceutical may have solubility, bioavailability or other properties suitable for disease treatment. Thus, the discovery and development of novel or beneficial polymorphs is extremely important, especially in the pharmaceutical area.

Amorphous solids, on the other hand, cannot be characterized according to habit or polymorphic form. An amorphous solid is in a high-energy structural state relative to its crystalline form which can give rise to instability problems. It may crystallize during storage or shipping or an amorphous solid may be more sensitive to oxidation (Pikal et al.,1997, J. Pharm. Sci. 66:1312). A common amorphous solid is glass in which the atoms and molecules exist in a nonuniform array. Amorphous solids are usually the result of rapid solidification and can be conveniently identified (but not characterized) by x-ray powder diffraction, since these solids give very diffuse lines or no crystal diffraction pattern.

Crystals are normally obtained by dissolving a compound in a suitable solvent and then adjusting the conditions to induce crystal growth. The crystallization process commonly involves dissolving the compound to saturation and then lowering the temperature. Upon cooling, the solution becomes supersaturated which often leads to the appearance of the crystals. Sometimes, crystal formation is induced by mechanically disturbing the solution, such as by scratching the inner surface of the solution container, or by seeding the solution with dust or crystals of the same compound. The pH, rate of cooling, type of solvent, solute-solvent ratio, additives such as surfactants, and inhibitors not only affect the purity of the crystals that form, but they may affect the crystal habit or polymorph that predominates. Other methods of crystal cultivation are sublimation, solvent evaporation, vapor diffusion, heating, crystallization from the melt, rapid pH change, thermal desolvation of crystalline solvates, and crystallization in the presence of additives (Guillory, Polymorphism in Pharmaceutical Solids, 186, 1999). Because of the extremely large number of possible combinations of components and experimental conditions, the range of conditions that may produce novel or known solid forms is very large, and locating optimal solid forms is commensurately difficult. The present invention can be used not only to determine conditions that produce optimal solid forms, but can also be used to determine conditions that produce solid forms of compounds that may be very difficult to crystallize under most (or typical) conditions.

As used herein, the term “array” means a plurality of samples, preferably, at least 24 samples each sample comprising a compound-of-interest and at least one component, wherein: (a) an amount of the compound-of-interest in each sample is less than about 100 milligrams; and (b) at least one of the samples comprises a solid-form of the compound-of-interest. An array can comprise 2 or more samples, for example, 24, 36, 48, 96, or more samples, preferably 1000 or more samples, more preferably, 10,000 or more samples. An array can comprise one or more groups of samples also known as sub-arrays. For example, a group can be a 96-vessel plate of sample vessels (such as sample tubes) or a 96-well plate of sample wells in an array consisting of 100 or more plates. Each sample or selected samples or each sample group of selected sample groups in the array can be subjected to the same or different processing parameters; each sample or sample group can have different components or concentrations of components; or both to induce, inhibit, prevent, or reverse formation of solid-forms of the compound-of-interest. Arrays can be prepared by preparing a plurality of samples, each sample comprising a compound-of-interest and one or more components, then processing the samples to induce, inhibit, prevent, or reverse formation of solid-forms of the compound-of-interest.

As used herein, the term “sample” means a mixture of a compound-of-interest and one or more additional components to be subjected to various processing parameters and then screened to detect the presence or absence of solid-forms, preferably, to detect desired solid-forms with new or enhanced properties. In addition to the compound-of-interest, the sample comprises one or more components, preferably, 2 or more components, or 3 or more components. Each additional component adds one or more additional degrees of freedom to the experiment, greatly increasing the number of possible experiments, and in some cases enhancing the ability of the informatics system to perform its functions. In general, a sample will comprise one compound-of-interest but can comprise multiple compounds-of-interest. Typically, a sample comprises less than about 1 g of the compound-of-interest, preferably, less than about 100 mg, more preferably, less than about 25 mg, even more preferably, less than about 1 mg, still more preferably less than about 100 micrograms, and optimally less than about 100 nanograms of the compound-of-interest. Preferably, the sample has a total volume of less than about 100 to about 250 ul.

As used herein, the term “pharmaceutical” means any substance that has a therapeutic, disease preventive, diagnostic, or prophylactic effect when administered to an animal or a human. The term pharmaceutical includes prescription pharmaceuticals and over the counter pharmaceuticals. Pharmaceuticals suitable for use in the invention include all those known or to be developed. A pharmaceutical can be a large molecule (i.e., molecules having a molecular weight of greater than about 1000 g/mol), such as oligonucleotides, polynucleotides, oligonucleotide conjugates, polynucleotide conjugates, proteins, peptides, peptidomimetics, or polysaccharides or small molecules (i.e., molecules having a molecular weight of less than about 1000 g/mol), such as hormones, steroids, nucleotides, nucleosides, or aminoacids.

Examples of suitable small molecule pharmaceuticals include, but are not limited to, cardiovascular pharmaceuticals, such as amlodipine, losartan, irbesartan, diltiazem, clopidogrel, digoxin, abciximab, furosemide, amiodarone, beraprost, tocopheryl; anti-infective components, such as amoxicillin, clavulanate, azithromycin, itraconazole, acyclovir, fluconazole, terbinafine, erythromycin, and acetyl sulfisoxazole; psychotherapeutic components, such as sertaline, vanlafaxine, bupropion, olanzapine, buspirone, alprazolam, methylphenidate, fluvoxamine, and ergoloid; gastrointestinal products, such as lansoprazole, ranitidine, famotidine, ondansetron, granisetron, sulfasalazine, and infliximab; respiratory therapies, such as loratadine, fexofenadine, cetirizine, fluticasone, salmeterol, and budesonide; cholesterol reducers, such as atorvastatin calcium, lovastatin, bezafibrate, ciprofibrate, and gemfibrozil; cancer and cancer-related therapies, such as paclitaxel, carboplatin, tamoxifen, docetaxel, epirubicin, leuprolide, bicalutamide, goserelin implant, irinotecan, gemcitabine, and sargramostim; blood modifiers, such as epoetin alfa, enoxaparin sodium, and antihemophilic factor; antiarthritic components, such as celecoxib, nabumetone, misoprostol, and rofecoxib; AIDS and AIDS-related pharmaceuticals, such as lamivudine, indinavir, stavudine, and lamivudine; diabetes and diabetes-related therapies, such as metformin, troglitazone, and acarbose; biologicals, such as hepatitis B vaccine, and hepatitis A vaccine; hormones, such as estradiol, mycophenolate mofetil, and methylprednisolone; analgesics, such as tramadol hydrochloride, fentanyl, metamizole, ketoprofen, morphine, lysine acetylsalicylate, ketoralac tromethamine, loxoprofen, and ibuprofen; dermatological products, such as isotretinoin and clindamycin; anesthetics, such as propofol, midazolam, and lidocaine hydrochloride; migraine therapies, such as sumatriptan, zolmitriptan, and rizatriptan; sedatives and hypnotics, such as zolpidem, zolpidem, triazolam, and hycosine butylbromide; imaging components, such as iohexol, technetium, TC99M, sestamibi, iomeprol, gadodiamide, ioversol, and iopromide; and diagnostic and contrast components, such as alsactide, americium, betazole, histamine, mannitol, metyrapone, petagastrin, phentolamine, radioactive B12, gadodiamide, gadopentetic acid, gadoteridol, and perflubron.

Other examples of suitable pharmaceuticals are listed in 2000 Med Ad News 19:56-60 and The Physicians Desk Reference, 53rd edition, 792-796, Medical Economics Company (1999), both of which are incorporated herein by reference.

Examples of suitable veterinary pharmaceuticals include, but are not limited to, vaccines, antibiotics, growth enhancing components, and dewormers. Other examples of suitable veterinary pharmaceuticals are listed in The Merck Veterinary Manual, 8th ed., Merck and Co., Inc., Rahway, N.J., 1998; (1997) The Encyclopedia of Chemical Technology, 24 Kirk-Othomer (4^{th} ed. at 826); and Veterinary Drugs in ECT 2nd ed., Vol 21, by A. L. Shore and R. J. Magee, American Cyanamid Co.

As used herein, the term “dietary supplement” means a non-caloric or insignificant-caloric substance administered to an animal or a human to provide a nutritional benefit or a non-caloric or insignificant-caloric substance administered in a food to impart the food with an aesthetic, textural, stabilizing, or nutritional benefit. Dietary supplements include, but are not limited to, fat binders, such as caducean; fish oils; plant extracts, such as garlic and pepper extracts; vitamins and minerals; food additives, such as preservatives, acidulents, anticaking components, antifoaming components, antioxidants, bulking components, coloring components, curing components, dietary fibers, emulsifiers, enzymes, firming components, humectants, leavening components, lubricants, non-nutritive sweeteners, food-grade solvents, thickeners; fat substitutes, and flavor enhancers; and dietary aids, such as appetite suppressants.

Examples of suitable dietary supplements are listed in (1994) The Encyclopedia of Chemical Technology, 11 Kirk-Othomer (4^{th} ed. at 805-833). Examples of suitable vitamins are listed in (1998) The Encyclopedia of Chemical Technology, 25 Kirk-Othomer (4^{th} ed. at 1) and Goodman & Gilman's: The Pharmacological Basis of Therapeutics, 9th Edition, eds. Joel G. Harman and Lee E. Limbird, McGraw-Hill, 1996 p.1547, both of which are incorporated by reference herein. Examples of suitable minerals are listed in The Encyclopedia of Chemical Technology, 16 Kirk-Othomer (4^{th} ed. at 746) and “Mineral Nutrients” in ECT 3rd ed., Vol 15, pp. 570-603, by C. L. Rollinson and M. G. Enig, University of Maryland, both of which are incorporated herein by reference

As used herein, the term “alternative medicine” means a substance, preferably a natural substance, such as a herb or an herb extract or concentrate, administered to a subject or a patient for the treatment of disease or for general health or well being, wherein the substance does not require approval by the FDA. Examples of suitable alternative medicines include, but are not limited to, ginkgo biloba, ginseng root, valerian root, oak bark, kava kava, echinacea, harpagophyti radix, others are listed in The Complete German Commission E Monographs: Therapeutic Guide to Herbal Medicine, Mark Blumenthal et al. eds., Integrative Medicine Communications 1998, incorporated by reference herein.

As used herein the term “nutraceutical” means a food or food product having both caloric value and pharmaceutical or therapeutic properties. Example of nutraceuticals include garlic, pepper, brans and fibers, and health drinks Examples of suitable Nutraceuticals are listed in M. C. Linder, ed. Nutritional Biochemistry and Metabolism with Clinical Applications, Elsevier, N.Y., 1985; Pszczola et al., 1998 Food technology 52:30-37 and Shukla et al., 1992 Cereal Foods World 37:665-666.

As used herein, the term “sensory-material” means any chemical or substance, known or to be developed, that is used to provide an olfactory or taste effect in a human or an animal, preferably, a fragrance material, a flavor material, or a spice. A sensory-material also includes any chemical or substance used to mask an odor or taste. Examples of suitable fragrances materials include, but are not limited to, musk materials, such as civetone, ambrettolide, ethylene brassylate, musk xylene, Tonalide®, and Glaxolide®; amber materials, such as ambrox, ambreinolide, and ambrinol; sandalwood materials, such as α-santalol, β-santalol, Sandalore®, and Bacdanol®; patchouli and woody materials, such as patchouli oil, patchouli alcohol, Timberol® and Polywood®; materials with floral odors, such as Givescone®, damascone, irones, linalool, Lilial®, Lilestralis®, and dihydrojasmonate. Other examples of suitable fragrance materials for use in the invention are listed in Perfumes: Art, Science, Technology, P. M. Muller ed. Elsevier, N.Y., 1991, incorporated herein by reference. Examples of suitable flavor materials include, but are not limited to, benzaldehyde, anethole, dimethyl sulfide, vanillin, methyl anthranilate, nootkatone, and cinnamyl acetate. Examples of suitable spices include but are not limited to allspice, tarrogon, clove, pepper, sage, thyme, and coriander. Other examples of suitable flavor materials and spices are listed in Flavor and Fragrance Materials-1989, Allured Publishing Corp. Wheaton, Ill., 1989; Bauer and Garbe Common Flavor and Fragrance Materials, VCH Verlagsgesellschaft, Weinheim, 1985; and (1994) The Encyclopedia of Chemical Technology, 11 Kirk-Othomer (4^{th} ed. at 1-61), all of which are incorporated by reference herein.

As used herein, the term “agrochemical” means any substance known or to be developed that is used on the farm, yard, or in the house or living area to benefit gardens, crops, ornamental plants, shrubs, or vegetables or kill insects, plants, or fungi. Examples of suitable agrochemicals for use in the invention include pesticides, herbicides, fungicides, insect repellants, fertilizers, and growth enhancers. For a discussion of agrochemicals see The Agrochemicals Handbook (1987) 2nd Edition, Hartley and Kidd, editors: The Royal Society of Chemistry, Nottingham, England.

Pesticides include chemicals, compounds, and substances administered to kill vermin such as bugs, mice, and rats and to repel garden pests such as deer and woodchucks. Examples of suitable pesticides that can be used according to the invention include, but are not limited to, abamectin (acaricide), bifenthrin (acaricide), cyphenothrin (insecticide), imidacloprid (insecticide), and prallethrin (insectide). Other examples of suitable pesticides for use in the invention are listed in Crop Protection Chemicals Reference, 6th ed., Chemical and Pharmaceutical Press, John Wiley & Sons Inc., New York, 1990; (1996) The Encyclopedia of Chemical Technology, 18 Kirk-Othomer (4^{th} ed. at 311-341); and Hayes et al., Handbook of Pesticide Toxicology, Academic Press, Inc., San Diego, Calif., 1990, all of which are incorporated by reference herein.

Herbicides include selective and non-selective chemicals, compounds, and substances administered to kill plants or inhibit plant growth. Examples of suitable herbicides include, but are not limited to, photosystem I inhibitors, such as actifluorfen; photosystem II inhibitors, such as atrazine; bleaching herbicides, such as fluridone and difunon; chlorophyll biosynthesis inhibitors, such as DTP, clethodim, sethoxydim, methyl haloxyfop, tralkoxydim, and alacholor; inducers of damage to antioxidative system, such as paraquat; amino-acid and nucleotide biosynthesis inhibitors, such as phaseolotoxin and imazapyr; cell division inhibitors, such as pronamide; and plant growth regulator synthesis and function inhibitors, such as dicamba, chloramben, dichlofop, and ancymidol. Other examples of suitable herbicides are listed in Herbicide Handbook, 6th ed., Weed Science Society of America, Champaign, Ill. 1989; (1995) The Encyclopedia of Chemical Technology, 13 Kirk-Othomer (4th ed. at 73-136); and Duke, Handbook of Biologically Active Phytochemicals and Their Activities, CRC Press, Boca Raton, Fla., 1992, all of which are incorporated herein by reference.

Fungicides include chemicals, compounds, and substances administered to plants and crops that selectively or non-selectively kill fungi. For use in the invention, a fungicide can be systemic or non-systemic. Examples of suitable non-systemic fungicides include, but are not limited to, thiocarbamate and thiurame derivatives, such as ferbam, ziram, thiram, and nabam; imides, such as captan, folpet, captafol, and dichlofluanid; aromatic hydrocarbons, such as quintozene, dinocap, and chloroneb; dicarboximides, such as vinclozolin, chlozolinate, and iprodione. Example of systemic fungicides include, but are not limited to, mitochondiral respiration inhibitors, such as carboxin, oxycarboxin, flutolanil, fenfuram, mepronil, and methfuroxam; microtubulin polymerization inhibitors, such as thiabendazole, fuberidazole, carbendazim, and benomyl; inhibitors of sterol biosynthesis, such as triforine, fenarimol, nuarimol, imazalil, triadimefon, propiconazole, flusilazole, dodemorph, tridemorph, and fenpropidin; and RNA biosynthesis inhibitors, such as ethirimol and dimethirimol; phopholipic biosynthesis inhibitors, such as ediphenphos and iprobenphos. Other examples of suitable fungicides are listed in Torgeson, ed., Fungicides: An Advanced Treatise, Vols. 1 and 2, Academic Press, Inc., New York, 1967 and (1994) The Encyclopedia of Chemical Technology, 12 Kirk-Othomer (4th ed. at 73-227), all of which are incorporated herein by reference.

As used herein, a “consumer formulation” means a formulation for consumer use, not intended to be absorbed or ingested into the body of a human or animal, comprising an active component. Preferably, it is the active component that is investigated as the compound-of-interest in the arrays and methods of the invention. Consumer formulations include, but are not limited to, cosmetics, such as lotions, facial makeup; antiperspirants and deodorants, shaving products, and nail care products; hair products, such as and shampoos, colorants, conditioners; hand and body soaps; paints; lubricants; adhesives; and detergents and cleaners.

As used herein an “industrial formulation” means a formulation for industrial use, not intended to be absorbed or ingested into the body of a human or animal, comprising an active component. Preferably, it is the active component of industrial formulation that is investigated as the compound-of-interest in the arrays and methods of the invention. Industrial formulations include, but are not limited to, polymers; rubbers; plastics; industrial chemicals, such as solvents, bleaching agents, inks, dyes, fire retardants, antifreezes and formulations for deicing roads, cars, trucks, jets, and airplanes; industrial lubricants; industrial adhesives; industrial enzymes; construction materials, such as cements.

One of skill in the art will readily be able to choose active and inactive components used in consumer and industrial formulations and set up arrays according to the invention. Such active components and inactive components are well known in the literature and the following references are provided merely by way of example. Active components and inactive components for use in cosmetic formulations are listed in (1993) The Encyclopedia of Chemical Technology, 7 Kirk-Othomer (4^{th} ed. at 572-619); M. G. de Navarre, The Chemistry and Manufacture of Cosmetics, D. Van Nostrand Company, Inc., New York, 1941; CTFA International Cosmetic Ingredient Dictionary and Handbook, 8th Ed., CTFA, Washington, D.C., 2000; and A. Nowak, Cosmetic Preparations, Micelle Press, London, 1991. All of which are incorporated by reference herein. Active components and inactive components for use in hair care products are listed in (1994) The Encyclopedia of Chemical Technology, 12 Kirk-Othomer (4^{th} ed. at 881-890) and Shampoos and Hair Preparations in ECT 1 st ed., Vol. 12, pp. 221-243, by F. E. Wall, both of which are incorporated by reference herein. Active components and inactive components for use in hand and body soaps are listed in (1997) The Encyclopedia of Chemical Technology, 22 Kirk-Othomer (4^{th} ed. at 297-396), incorporated by reference herein. Active components and inactive components for use in paints are listed in (1996) The Encyclopedia of Chemical Technology, 17 Kirk-Othomer (4^{th} ed. at 1049-1069) and “Paint” in ECT 1st ed., Vol. 9, pp. 770-803, by H. E. Hillman, Eagle Paint and Varnish Corp, both of which are incorporated by reference herein. Active components and inactive components for use in consumer and industrial lubricants are listed in (1995) The Encyclopedia of Chemical Technology, 15 Kirk-Othomer (4^{th} ed. at 463-517); D. D. Fuller, Theory and practice of Lubrication for Engineers, 2nd ed., John Wiley & Sons, Inc., 1984; and A. Raimondi and A. Z. Szeri, in E. R. Booser, eds., Handbook of Lubrication, Vol. 2, CRC Press Inc., Boca Raton, Fla., 1983, all of which are incorporated by reference herein. Active components and inactive components for use in consumer and industrial adhesives are listed in (1991) The Encyclopedia of Chemical Technology, 1 Kirk-Othomer (4^{th} ed. at 445-465) and I. M. Skeist, ed. Handbook of Adhesives, 3rd ed. Van Nostrand-Reinhold, New York, 1990, both of which are incorporated herein by reference. Active components and inactive components for use in polymers are listed in (1996) The Encyclopedia of Chemical Technology, 19 Kirk-Othomer (4^{th} ed. at 881-904), incorporated herein by reference. Active components and inactive components for use in rubbers are listed in (1997) The Encyclopedia of Chemical Technology, 21 Kirk-Othomer (4^{th} ed. at 460-591), incorporated herein by reference. Active components and inactive components for use in plastics are listed in (1996) The Encyclopedia of Chemical Technology, 19 Kirk-Othomer (4^{th} ed. at 290-316), incorporated herein by reference. Active components and inactive components for use with industrial chemicals are listed in Ash et al., Handbook of Industrial Chemical Additives, VCH Publishers, New York 1991, incorporated herein by reference. Active components and inactive components for use in bleaching components are listed in (1992) The Encyclopedia of Chemical Technology, 4 Kirk-Othomer (4^{th} ed. at 271-311), incorporated herein by reference. Active components and inactive components for use inks are listed in (1995) The Encyclopedia of Chemical Technology, 14 Kirk-Othomer (4^{t}h ed. at 482-503), incorporated herein by reference. Active components and inactive components for use in dyes are listed in (1993) The Encyclopedia of Chemical Technology, 8 Kirk-Othomer (4^{th} ed. at 533-860), incorporated herein by reference. Active components and inactive components for use in fire retardants are listed in (1993) The Encyclopedia of Chemical Technology, 10 Kirk-Othomer (4^{th} ed. at 930-1022), incorporated herein by reference. Active components and inactive components for use in antifreezes and deicers are listed in (1992) The Encyclopedia of Chemical Technology, 3 Kirk-Othomer (4^{th} ed. at 347-367), incorporated herein by reference. Active components and inactive components for use in cement are listed in (1993) The Encyclopedia of Chemical Technology, 5 Kirk-Othomer (4^{th} ed. at 564), incorporated herein by reference.

As used herein, the term “component” means any substance that is combined, mixed, or processed with the compound-of-interest to form a sample or impurities, for example, trace impurities left behind after synthesis or manufacture of the compound-of-interest. The term component includes solvents in the sample. The term component also encompasses the compound-of-interest itself. The compound-of-interests to be screened can be any useful compound including, but not limited to, pharmaceuticals, dietary supplements, nutraceuticals, agrochemicals, or alternative medicines. The invention is particularly well-suited for screening solid-forms of a single low-molecular-weight organic molecules. Thus, the invention encompasses arrays of diverse solid-forms of a single low-molecular-weight molecule.

A single substance can exist in one or more physical states having different properties thereby classified herein as different components. For instance, the amorphous and crystalline forms of an identical compound are classified as different components. Components can be large molecules (i.e., molecules having a molecular weight of greater than about 1000 g/mol), such as large-molecule pharmaceuticals, oligonucleotides, polynucleotides, oligonucleotide conjugates, polynucleotide conjugates, proteins, peptides, peptidomimetics, or polysaccharides or small molecules (i.e., molecules having a molecular weight of less than about 1000 g/mol) such as small-molecule pharmaceuticals, hormones, nucleotides, nucleo sides, steroids, or amino acids.

Components can also be chiral or optically-active substances or compounds, such as optically-active solvents, optically-active reagents, or optically-active catalysts. Preferably, components promote or inhibit or otherwise effect precipitation, formation, crystallization, or nucleation of solid-forms, preferably, solid-forms of the compound-of-interest. Thus, a component can be a substance whose intended effect in an array sample is to induce, inhibit, prevent, modify, or reverse formation of solid-forms of the compound-of-interest. Examples of components include, but are not limited to, excipients; solvents; salts; acids; bases; gases; small molecules, such as hormones, steroids, nucleotides, nucleosides, and aminoacids; large molecules, such as oligonucleotides, polynucleotides, oligonucleotide and polynucleotide conjugates, proteins, peptides, peptidomimetics, and polysaccharides; pharmaceuticals; dietary supplements; alternative medicines; nutraceuticals; sensory compounds; agrochemicals; the active component of a consumer formulation; and the active component of an industrial formulation; crystallization additives, such as additives that promote and/or control nucleation, additives that affect crystal habit, and additives that affect polymorphic form; additives that affect particle or crystal size; additives that structurally stabilize crystalline or amorphous solid-forms; additives that dissolve solid-forms; additives that inhibit crystallization or solid formation; optically-active solvents; optically-active reagents; optically-active catalysts; and even packaging or processing reagents.

Components include acidic substances and basic substances. Such substances can react to form a salt with the compound-of-interest or other components present in a sample. When a salt of the compound-of-interest is desired, salt forming components will generally be used in stoichiometric quantities. Components that are basic in nature are capable of forming a wide variety of salts with various inorganic and organic acids. For example, suitable acids are those that form the following salts with basic compounds: chloride, bromide, iodide, acetate, salicylate, benzenesulfonate, benzoate, bicarbonate, bitartrate, calcium edetate, camsylate, carbonate, citrate, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide, and pamoate (i.e., 1,1′-methylene-bis-(2-hydroxy-3-naphthoate)). Components that include an amino moiety also can form pharmaceutically-acceptable salts with various amino acids, in addition to the acids mentioned above.

The term “excipient” as used herein refers to substances used to formulate actives into pharmaceutical formulations. Preferably, an excipient does not lower or interfere with the primary therapeutic effect of the active, more preferably, an excipient is therapeutically inert. The term “excipient” encompasses carriers, solvents, diluents, vehicles, stabilizers, and binders. Excipients can also be those substances present in a pharmaceutical formulation as an indirect result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

Examples of suitable excipients include, but are not limited to, acidulents, such as lactic acid, hydrochloric acid, and tartaric acid; solubilizing components, such as non-ionic, cationic, and anionic surfactants; absorbents, such as bentonite, cellulose, and kaolin; alkalizing components, such as diethanolamine, potassium citrate, and sodium bicarbonate; anticaking components, such as calcium phosphate tribasic, magnesium trisilicate, and talc; antimicrobial components, such as benzoic acid, sorbic acid, benzyl alcohol, benzethonium chloride, bronopol, alkyl parabens, cetrimide, phenol, phenylmercuric acetate, thimerosol, and phenoxyethanol; antioxidants, such as ascorbic acid, alpha tocopherol, propyl gallate, and sodium metabisulfite; binders, such as acacia, alginic acid, carboxymethyl cellulose, bydroxyethyl cellulose; dextrin, gelatin, guar gum, magnesium aluminum silicate, maltodextrin, povidone, starch, vegetable oil, and zein; buffering components, such as sodium phosphate, malic acid, and potassium citrate; chelating components, such as EDTA, malic acid, and maltol; coating components, such as adjunct sugar, cetyl alcohol, polyvinyl alcohol, camauba wax, lactose maltitol, titanium dioxide; controlled release vehicles, such as microcrystalline wax, white wax, and yellow wax; desiccants, such as calcium sulfate; detergents, such as sodium lauryl sulfate; diluents, such as calcium phosphate, sorbitol, starch, talc, lactitol, polymethacrylates, sodium chloride, and glyceryl palmitostearate; disintegrants, such as collodial silicon dioxide, croscarmellose sodium, magnesium aluminum silicate, potassium polacrilin, and sodium starch glycolate; dispersing components, such as poloxamer 386, and polyoxyethylene fatty esters (polysorbates); emollients, such as cetearyl alcohol, lanolin, mineral oil, petrolatum, cholesterol, isopropyl myristate, and lecithin; emulsifying components, such as anionic emulsifying wax, monoethanolamine, and medium chain triglycerides; flavoring components, such as ethyl maltol, ethyl vanillin, fumaric acid, malic acid, maltol, and menthol; humectants, such as glycerin, propylene glycol, sorbitol, and triacetin; lubricants, such as calcium stearate, canola oil, glyceryl palmitosterate, magnesium oxide, poloxymer, sodium benzoate, stearic acid, and zinc stearate; solvents, such as alcohols, benzyl phenylformate, vegetable oils, diethyl phthalate, ethyl oleate, glycerol, glycofurol, for indigo carmine, polyethylene glycol, for sunset yellow, for tartazine, triacetin; stabilizing components, such as cyclodextrins, albumin, xanthan gum; and tonicity components, such as glycerol, dextrose, potassium chloride, and sodium chloride; and mixture thereof. Other examples of suitable excipients, such as binders and fillers are listed in Remington's Pharmaceutical Sciences, 18th Edition, ed. Alfonso Gennaro, Mack Publishing Co. Easton, Pa., 1995 and Handbook of Pharmaceutical Excipients, 3rd Edition, ed. Arthur H. Kibbe, American Pharmaceutical Association, Washington D.C. 2000, both of which are incorporated herein by reference.

In general, the arrays of the invention will contain a solvent as one on the components. Solvents may influence and direct the formation of solid-forms through polarity, viscosity, boiling point, volatility, charge distribution, and molecular shape. The solvent identity and concentration is one way to control saturation. Indeed, one can crystallize under isothermal conditions by simply adding a nonsolvent to an initially subsaturated solution. One can start with an array of a solution of the compound-of-interest in which varying amounts of nonsolvent are added to each of the individual elements of the array. The solubility of the compound is exceeded when some critical amount of nonsolvent is added. Further addition of the nonsolvent increases the supersaturation of the solution and, therefore, the growth rate of the crystals that are grown.

As used herein, the term “experimental parameters” means the physical or chemical conditions under which a sample is subjected and the time during which the sample is subjected to such conditions. Experimental parameters include, but are not limited to, the temperature, time, pH, amount or the concentration of a component, component identity, solvent removal rate, and solvent composition. Sub-arrays or even individual samples within an array can be subjected to processing parameters that are different from the processing parameters to which other sub-arrays or samples, within the same array, are subjected. Processing parameters will differ between sub-arrays or samples when they are intentionally varied to induce a measurable change in the sample's properties. Thus, according to the invention, minor variations, such as those introduced by slight adjustment errors, are not considered intentionally varied.

When referring to an interaction between components, an “interaction” means that the components as a mixture display a property (e.g., the ability to solubilize a specific pharmaceutical) of a different magnitude or value than the same property displayed by each component in isolation. Interactions between components will affect the properties of samples. Merely for example, a particular combination and ratio of excipients can interact such that the combination has a high solubilizing power for a particular pharmaceutical. Once such an interaction is detected, it can be exploited to develop enhanced formulations for the pharmaceutical.

As used herein, the term “property” means a structural, physical, pharmacological, or chemical characteristic of a sample, preferably, a structural, physical, pharmacological, or chemical characteristics of a compound-of-interest. The properties of a sample, as well as the interactions or the manifestations or outcomes of those interactions arising from or involving the original sample, can be analyzed using methods or techniques known in the art. Some examples of these methods or techniques are Raman and infrared spectroscopy, ultraviolet spectroscopy, x-ray diffraction, scanning electron microscopy, transmission electron microscopy, near field scanning optical microscopy, far field scanning optical microscopy, atomic force microscopy, micro-thermal analysis, differential analyis, nuclear magnetic resonance spectroscopy, gas chromatography, and high-pressure or high-performance liquid chromatography.

Preferred properties are those that relate to the efficacy, safety, stability, or utility of the compound-of-interest or a formulation thereof. For example, regarding pharmaceutical, dietary supplement, alternative medicine, and nutraceutical compounds and substances, properties include physical properties, such as stability, solubility, dissolution, permeability, and partitioning; mechanical properties, such as compressibility, compactability, and flow characteristics; the formulation's sensory properties, such as color, taste, and smell; and properties that affect the utility, such as absorption, bioavailability, toxicity, metabolic profile, and potency. Other properties include those which affect the compound-of-interest's behavior and ease of processing in a crystallizer or a formulating machine. For a discussion of industrial crystallizers and properties thereof see (1993) The Encyclopedia of Chemical Technology, 7 Kirk-Othomer (4^{th} ed. pp. 720-729). Such processing properties are closely related to the solid-form's mechanical properties and its physical state, especially degree of agglomeration. Concerning pharmaceuticals, dietary supplements, alternative medicines, and nutraceuticals, optimizing physical and utility properties of their solid-forms can result in a lowered required dose for the same therapeutic effect. Thus, there are potentially fewer side effects that can improve patient compliance.

Structural properties include, but are not limited to, whether the compound-of-interest can be crystallize, whether it is solid, and if solid, is it crystalline or amorphous, and if crystalline, the polymorphic form and a description of the crystal habit. Structural properties also include the composition, such as whether the solid-form is a hydrate, solvate, or a salt. Examples of structural property are surface-to-volume ratio and the degree of agglomeration of the particles. Surface-to-volume ratio decreases with the degree of agglomeration. It is well known that a high surface-to-volume ratio improves the solubility rate. Small-size particles have high surface-to-volume ratio. The surface-to-volume ratio is also influenced by the crystal habit, for example, the surface-to-volume ratio increases from spherical shape to needle shape to dendritic shape. Porosity also affects the surface-to-volume ratio, for example, solid-forms having channels or pores (e.g., inclusions, such as hydrates and solvates) have a high surface-to-volume ratio.

Still another structural property is particle size and particle-size distribution. For example, depending on concentrations, the presence of inhibitors or impurities, and other conditions, particles can form from solution in different sizes and size distributions. Particulate matter, produced by precipitation or crystallization, has a distribution of sizes that varies in a definite way throughout the size range. Particle- and crystal-size distribution is generally expressed as a population distribution relating to the number of particles at each size. In pharmaceuticals, particle and crystal size distribution have very important clinical aspects, such as bioavailability. Thus, compounds or compositions that promote small crystal size can be of clinical importance.

Physical properties include, but are not limited to, physical stability, melting point, solubility, strength, hardness, compressibility, and compactability. Physical stability refers to a compound's or composition's ability to maintain its physical form, for example maintaining particle size; maintaining crystal or amorphous form; maintaining complexed form, such as hydrates and solvates; resistance to absorption of ambient moisture; and maintaining of mechanical properties, such as compressibility and flow characteristics. Methods for measuring physical stability include spectroscopy, sieving or testing, microscopy, sedimentation, stream scanning, and light scattering. Polymorphic changes, for example, are usually detected by differential scanning calorimetry or quantitative infrared analysis. For a discussion of the theory and methods of measuring physical stability see Fiese et al., in The Theory and Practice of Industrial Pharmacy, 3rd ed., Lachman L.; Lieberman, H. A.; and Kanig, J. L. Eds., Lea and Febiger, Philadelphia, 1986 pp. 193-194 and Remington's Pharmaceutical Sciences, 18th Edition, ed. Alfonso Gennaro, Mack Publishing Co. Easton, Pa., 1995, pp. 1448-1451, both of which are incorporated herein by reference.

Solubility refers to the equilibrium solubility or steady state and is measured as weight component/volume solvent. When an active component, such as a pharmaceutical substance has an aqueous solubility of less than about 1 milligram/milliliter in the physiological pH range of 1-7, a potential bioavailability problem exists. Descriptive terms used to describe solubility given in parts of solvent for 1 part of solute are: very soluble (<1 part); freely soluble (from 1 to 10 parts); soluble (from 10 to 30 parts); sparingly soluble (from 30 to 100 parts); slightly soluble (from 100 to 1,000 parts); very slightly soluble (from 1,000 to 10,000 parts); and insoluble (>10,000 parts). For a discussion of solution and phase equilibria see Remington's Pharmaceutical Sciences, 18th Edition, ed. Alfonso Gennaro, Mack Publishing Co. Easton, Pa., 1995, Ch. 16, incorporated herein by reference.

The solubility can be tested by mixing the sample with a test solvent and agitating the sample at a constant temperature until equilibrium is achieved. Equilibrium usually occurs upon agitating the samples for 6 to 24 hours. If the component is acidic or basic, its solubility can be influenced by pH and one of skill in the art will take such factors into consideration when testing the solubility properties of a sample. Once equilibrium has occurred, the sample can be tested to determine the amount of component dissolved using standard technology, such as mass spectroscopy, HPLC, UV spectroscopy, fluorescence spectroscopy, gas chromatography, optical density, or by colorimetery. For a discussion of the theory and methods of measuring solubility see Streng et al., 1984 J. Pharm. Sci. 63:605; Kaplan 1972 Drug Metab. Rev. 1:15; and Remington's Pharmaceutical Sciences, 18th Edition, ed. Alfonso Gennaro, Mack Publishing Co. Easton, Pa., 1995, pp.1456-1457, all three of which are incorporated herein by reference. For a discussion of heat of dissolution, pKa, and pH solubility profile effects and techniques for measurement thereof see Fiese et al., in The Theory and Practice of Industrial Pharmacy, 3rd ed., Lachman L.; Lieberman, H. A.; and Kanig, J. L. Eds., Lea and Febiger, Philadelphia, 1986 pp. 185-188, incorporated herein by reference.

Dissolution refers to the rate at which a solid enters into solution. Several factors affect dissolution such as solubility, particle size, crystalline state, and the presence of diluents, disintegrants, or other excipients. For a discussion of the theory and methods of measuring dissolution see Remington's Pharmaceutical Sciences, 18th Edition, ed. Alfonso Gennaro, Mack Publishing Co. Easton, Pa., 1995, Chapter 34, incorporated herein by reference.

Chemical properties include, but are not limited to chemical stability, such as susceptibility to oxidation and reactivity with other compounds, such as acids, bases, or chelating agents. Chemical stability refers to resistance to chemical reactions induced, for example, by heat, ultraviolet radiation, moisture, chemical reactions between components, or oxygen. Well known methods for measuring chemical stability include mass spectroscopy, UV-VIS spectroscopy, HPLC, gas chromatography, and liquid chromatography-mass spectroscopy (LC-MS). For a discussion of the theory and methods of measuring chemical stability see Xu et al., Stability-Indicating HPLC Methods for Drug Analysis American Pharmaceutical Association, Washington D.C. 1999 and Remington's Pharmaceutical Sciences, 18th Edition, ed. Alfonso Gennaro, Mack Publishing Co. Easton, Pa., 1995, pp. 1458-1460, both of which are incorporated herein by reference.

As used herein, the term “solid-form” means a form of a solid substance, element, or chemical compound that is defined and differentiated from other solid-forms according to its physical state and properties.

The basic requirements for array and sample preparation and screening thereof are: (1) manually or electronically designing the experiment; (2) a distribution mechanism to add components and the compound-of-interest to separate sites, for example, on an array plate having sample wells or sample vessels. Preferably, the experiment design is performed electroncially using computer software, and the distribution mechanism is automated and controlled by computer software, which can optionally be linked to the experimental design software, and can vary at least one addition variable, e.g., the identity of the component(s) and/or the component concentration, more preferably, two or more variables. Such material handling technologies and robotics are well known to those skilled in the art. If desired, individual components can be placed at the appropriate sample site manually. This pick and place technique is also known to those skilled in the art. And (3) a screening mechanism to test each sample to detect a change in physical state or for one or more properties. Preferably, the testing mechanism is automated and driven by a computer. Preferably, the system further comprises a processing mechanism to process the samples after component addition. Optionally, the system can have a processing station the process the samples after preparation.

As used herein, “automated experimentation apparatus” and cognates thereof means a high-throughput apparatus for performing large numbers of experiments having at least one experimental step performed by computer-controlled apparatus. Human operators may direct the apparatus, or manually perform some portions of the process (e.g. moving groups of plates from one automated station to another, or performing an experimental procedure on results identified using a computer). “Fully automated experimentation apparatus” and cognates thereof means a high-throughput apparatus for performing large numbers of experiments in which all experimental steps are performed by computer-controlled apparatus. “High throughput experimentation apparatus” and cognates thereof means an apparatus for performing at least two simultaneous experiments.

As used herein, “high-throughput solid-form screening” means: performing a method for screening a plurality of solid-forms of a compound-of-interest, the method comprising the steps of (a) preparing at least 24 samples, each sample comprising the compound-of-interest and one or more components, wherein an amount of the compound-of-interest in each sample is less than about 1 gram; (b) processing at least 24 of the samples to generate an array wherein at least two of the processed samples comprise a solid-form of the compound-of-interest; and (c) analyzing the processed samples to detect at least one solid-form. Preferably, one or more experiments are performed to characterize at least one detected solid form.

As used herein, “high-throughput formulation screening” means: performing a method to (1) measure or detect an interaction between components; or (2) test or optimize one or more properties of a formulation of an active-component;
the method comprising the steps of:
   (a) preparing an array of samples, each sample comprising a component-in-common and at least one additional component, wherein each sample differs from a plurality of other samples with respect to at least one of:
      (i) the identity of the at least one additional component,
      (ii) the ratio of the component-in-common to the additional component, or
      (iii) the physical state of the component-in-common; and
   (b) testing each sample for one or more properties.

As used herein “model” means a computational entity that accepts as inputs data representing values of experimental parameters and/or results and produces as output data representing an estimate of one or more properties expected to result from an experiment corresponding to the input.

As used herein, the terms “compound” or “compound-of-interest” include, but are not limited to, pharmaceuticals, dietary supplements, alternative medicines, nutraceuticals, sensory compounds, agrochemicals, the active component of a consumer formulation, and the active component of an industrial formulation. In one preferred embodiment, the compound or compound-of-interest is a pharmaceutical.

A number of companies have developed array systems that can be adapted for use in the invention disclosed herein. Such systems may require modification, which is well within ordinary skill in the art. Examples of companies having array systems include Gene Logic of Gaithersburg, Md. (see U.S. Pat. No. 5,843,767 to Beattie), Luminex Corp., Austin, Tex., Beckman Instruments, Fullerton, Calif., MicroFab Technologies, Plano, Tex., Nanogen, San Diego, Calif., and Hyseq, Sunnyvale, Calif. These devices test samples based on a variety of different systems. All include thousands of microscopic channels that direct components into test wells, where reactions can occur. These systems are connected to computers for analysis of the data using appropriate software and data sets. The Beckman Instruments system can deliver nanoliter samples of 96 or 384-arrays, and is particularly well suited for hybridization analysis of nucleotide molecule sequences. The MicroFab Technologies system delivers sample using inkjet printers to aliquot discrete samples into wells. These and other systems can be adapted as required for use herein. For example, the combinations of the compound-of-interest and various components at various concentrations and combinations can be generated using standard formulating software (e.g., Matlab software, commercially available from Mathworks, Natick, Mass.). The combinations thus generated can be downloaded into a spread sheet, such as Microsoft EXCEL or stored in a relational database. A work list can be generated for instructing the automated distribution mechanism to prepare an array of samples according to the various combinations generated by the formulating software.

The work list can be generated using standard programming methods according to the automated distribution mechanism that is being used. The use of so-called work lists simply allows a file to be used as the process command rather than discrete programmed steps. The work list combines the formulation output of the formulating program with the appropriate commands in a file format directly readable by the automatic distribution mechanism. The automated distribution mechanism delivers at least one compound-of-interest, such as a pharmaceutical, as well as various additional components, such as solvents and additives, to each sample well. Preferably, the automated distribution mechanism can deliver multiple amounts of each component. Automated liquid and solid distribution systems are well known and commercially available, such as the Tecan Genesis, from Tecan-US, RTP, North Carolina. The robotic arm can collect and dispense the solutions, solvents, additives, or compound-of-interest form the stock plate to a sample well or sample vessel. The process is repeated until array is completed, for example, generating an array that moves from wells at left to right and from top to bottom in increasing polarity or non-polarity of solvent. Alternatively, it is often appropriate to randomize the positions of the samples in the array rather than placing them in order. The samples are then mixed. For example, the robotic arm moves up and down in each well plate for a set number of times to ensure proper mixing.

Liquid handling devices manufactured by vendors such as Tecan, Hamilton and Advanced Chemtech are all capable of being used in the invention. A prerequisite for all liquid handling devices is the ability to dispense to a sealed or sealable reaction vessel and have chemical compatibility for a wide range of solvent properties. The liquid handling device specifically manufactured for organic syntheses are the most desirable for application to crystallization due to the chemical compatibility issues. Robbins Scientific manufactures the Flexchem reaction block which consists of a Teflon reaction block with removable gasketed top and bottom plates. This reaction block is in the standard footprint of a 96-well microtiter plate and provides for individually sealed reaction chambers for each well. The gasketing material is typically Viton, neoprene/Viton, or Teflon coated Viton, and acts as a septum to seal each well. As a result, the pipetting tips of the liquid handling system need to have septum-piercing capability. The Flexchem reaction vessel is designed to be reusable in that the reaction block can be cleaned and reused with new gasket material.

An array can be prepared, processed, and screened as follows. The first step comprises selecting the component sources, preferably, at one or more concentrations. Preferably, at least one component source can deliver a compound-of-interest and one can deliver a solvent. Next, adding the compound-of-interest and components to a plurality of sample sites, such as sample wells or sample vessels on a sample plate to give an array of unprocessed samples. The array can then be processed according to the purpose and objective of the experiment, and one of skill in the art will readily ascertain the appropriate processing conditions. Preferably, the automated distribution mechanism as described above is used to distribute or add components.

Once an array containing samples comprising supersaturated solutions is prepared, solid formation can be induced by introducing a nucleation or precipitation event. In general, this involves subjecting a supersaturated solution to some form of energy, such as ultrasound or mechanical stimulation or by inducing supersaturation by adding additional components. Preferably, however, actively inducing solid formation is not required, and solid formation occurs spontaneously with the passage of time and/or changes in temperature.

The array can be processed according to the design and objective of the experiment. One of skill in the art will readily ascertain the appropriate processing conditions. Processing includes mixing; agitating; heating; cooling; adjusting the pressure; adding additional components, such as crystallization aids, nucleation promoters, nucleation inhibitors, acids, or bases, etc.; stirring; milling; filtering; centrifuging, emulsifying, subjecting one or more of the samples to mechanical stimulation; ultrasound; or laser energy; or subjection the samples to temperature gradient or simply allowing the samples to stand for a period of time at a specified temperature. A few of the more important processing parameters are elaborated below.

In some array experiments, processing will comprise dissolving either the compound-of-interest or one or more components. Solubility is commonly controlled by the composition (identity of components and/or the compound-of-interest) or by the temperature. The latter is most common in industrial crystallizers where a solution of a substance is cooled from a temperature at which it is in solution to one at which the solubility is exceeded. For example, the array can be processed by heating to a temperature (T1), preferably to a temperature at which the all the solids are completely in solution. The samples are then cooled, to a lower temperature (T2). The presence of solids can then determined. Implementation of this approach in arrays can be done on an individual sample site basis or for the entire array (i.e., all the samples in parallel). For example, each sample site could be warmed by local heating to a point at which the components and the compound-of-interest are dissolved. This step is followed by cooling through local thermal conduction or convection. A temperature sensor in each sample site can be used to record the temperature when the first crystal or precipitate is detected. In one embodiment, all the sample sites are processed individually with respect to temperature and small heaters, cooling coils, and temperature sensors for each sample site are provided and controlled. This approach is useful if each sample site has the same composition and the experiment is designed to sample a large number of temperature profiles to find those profiles that produce desired solid-forms. In another embodiment, the composition of each sample site is controlled and the entire array is heated and cooled as a unit. The advantage of the latter approach is that much simpler heating, cooling, and controlling systems can be utilized. Alternatively, thermal profiles are investigated by simultaneous experiments on identical array stages. Thus, a high-throughput matrix of experiments in both composition and thermal profiles can be obtained by parallel operation.

Typically, several distinct temperatures are tested during crystal nucleation and growth phases. Temperature can be controlled in either a static or dynamic manner. Static temperature means that a set incubation temperature is used throughout the experiment. Alternatively, a temperature gradient can be used. For example, the temperature can be lowered at a certain rate throughout the experiment. Furthermore, temperature can be controlled in a way as to have both static and dynamic components. For example, a constant temperature (e.g., 60° C.) is maintained during the mixing of crystallization reagents. After mixing of reagents is complete, controlled temperature decline is initiated (e.g., 60° C. to about 25° C. over 35 minutes).

Stand-alone devices employing Peltier-effect cooling and joule-heating are commercially available for use with microtiter plate footprints. A standard thermocycler used for PCR, such as those manufactured by MJ Research or PE Biosystems, can also be used to accomplish the temperature control. The use of these devices, however, necessitates the use of conical vials of conical bottom micro-well plates. If greater throughput or increased user autonomy is required, then full-scale systems such as the advanced Chemtech Benchmark Omega 96TM or Venture 596TM would be the platforms of choice. Both of these platforms utilize 96-well reaction blocks made from Teflon™. These reaction blocks can be rapidly and precisely controlled from −70 to 150° C. with complete isolation between individual wells. Also, both systems operate under inert atmospheres of nitrogen or argon and utilize all chemically inert liquid handling elements. The Omega 496 system has simultaneous independent dual coaxial probes for liquid handling, while the Venture 596 system has 2 independent 8-channel probe heads with independent z-control. Moreover, the Venture 596 system can process up to 10,000 reactions simultaneously. Both systems offer complete autonomy of operation.

Array samples can be incubated for various lengths of time (e.g., 5 minutes, 60 minutes, 48 hours, etc.). Since phase changes can be time dependent, it can be advantageous to monitors arrays experiments as a function of time. In many cases, time control is very important, for example, the first solid-form to crystallize may not be the most stable, but rather a metastable form which can then convert to a form stable over a period of time. This process is called “ageing”. Ageing also can be associated with changes in crystal size and/or habit. This type of ageing phenomena is called Ostwald ripening.

The pH of the sample medium can determine the physical state and properties of the solid phase that is generated. The pH can be controlled by the addition of inorganic and organic acids and bases. The pH of samples can be monitored with standard pH meters modified according to the volume of the sample.

The following discussion describes a number of preferred embodiments of the present invention, no part of which should be construed as limiting the present invention in any way.

In one preferred embodiment of the present invention, the system is used in conjunction with one or more high-throughput automated experimentation apparatus, such as Transform Pharmaceutical's FAST™ formulation system or CRYSTALMAX™ crystal discovery system. The FAST and CRYSTALMAX systems are described in U.S. patent applications Ser. Nos. 09/628,667 and 09/756,092, respectively, (the FAST™ and CRYSTALMAX™ applications) which are incorporated herein by reference. Words used herein are intended to be consistent with the FAST™ and CRYSTALMAX™ applications. In this embodiment, the system is used to plan and assess experiments performed with the CRYSTALMAX™ and FAST™ systems.

This embodiment includes a process informatics subsystem for controlling and acquiring data from the CRYSTALMAX and FAST systems, and a computational informatics subsystem for performing data mining, simulation, molecular modeling, high-dimensional multivariate visualizations of data, data clustering, categorizations, and other data processing. These subsystems operate on a shared database system used to store experimental results and analyses, as well as data derived from sources other than the process informatics subsystem, such as external databases and literature.

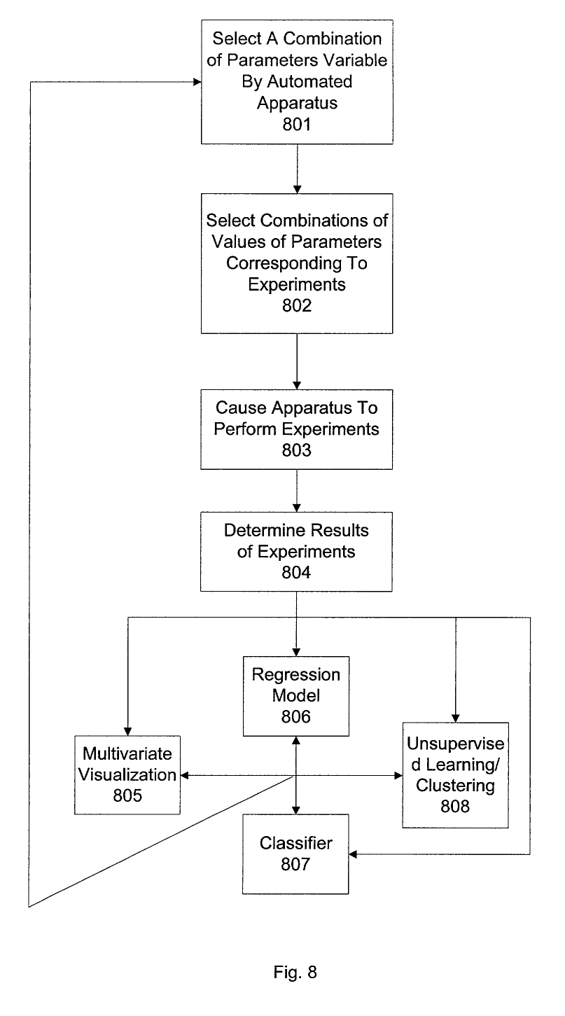

As schematically illustrated in FIG. 8, using the computational informatics subsystem, a combination of experimental parameters which may be varied by an automated experimentation apparatus such as FAST or CRYSTALMAX is selected 801. A plurality of distinct combinations of values of the experimental parameters is then determined, each combination corresponding to a distinct experiment 802. Using the process informatics subsystem, the automated experimentation apparatus is caused to conduct a set of experiments, each experiment corresponding to a distinct combination of the plurality of distinct combinations 803. The process informatics subsystem is also used to determine a collection of experimental results of the first set of experiments, the collection comprising a plurality of individual result sets, where each individual result set corresponds to a distinct experiment 804.

The experimental results determined in the methods of the present invention may be obtained using one or more of the following techniques: spectroscopy; sieving or testing; microscopy; optical imaging; sedimentation; stream scanning; light scattering; differential scanning calorimetry; infrared spectroscopy; quantitative infrared analysis; x-ray diffraction or x-ray powder diffraction; or Raman spectroscopy, including dispersive Raman spectroscopy and Fourier transform Raman or FT-Raman spectroscopy. Raman microscopes are available from a number of commercial sources, including, for example, the ALMEGA™ Dispersive Raman and the FT-Raman 960 (both available from Thermo Nicolet Corporation, Madison, Wis., USA).

The first collection of experimental results is processed through the computational informatics subsystem to determine a second combination of parameters variable by the automated experimentation apparatus 801, and a second plurality of distinct combinations of values of the experimental parameters 802, each combination of the second plurality corresponding to a distinct experiment. This process preferably may be iterated indefinitely to yield a third, fourth, fifth, or arbitrary number of subsequent pluralities of distinct combinations of experimental parameters, each combination corresponding to a distinct experiment. Although each combination preferably corresponds to a distinct experiment, in some circumstances multiples of each experiment are preferably performed to provide reliable data, particularly in stochastic processes such as crystallization. For crystallization experiments, preferably at least three experiments are performed for each distinct combination of experimental parameters, unless the quantity of the compound-of-interest makes three or more infeasible. When the quantity of available compound-of-interest is limited, it is often more desirable to perform more experiments corresponding to more distinct combinations of experimental parameters than to perform multiples. Further, when the amount of compound-of-interest is limited, it is generally desirable to perform fewer parallel experiments in a larger number of iterations (e.g. 100 iterations of 50 samples each, rather than 10 iterations of 500 samples) To determine combinations of parameters and values of the parameters, one or more multivariate visualizations 805, generated models 806 and 807, and/or unsupervised learning or clustering methods 808 are preferably employed. Generated models preferably comprise one or more regression model 806 and/or one or more classification model 807. A classification model takes one or more inputs and provides at least one class assignment as an output. A regression model takes one or more inputs and provides at least one output representing a variable that has a continuous range (e.g. at least one real or complex interval). The foregoing are preferably employed in combination, for example, a multivariate visualization of the results of a clustering calculation may be used to determine a classifier, as described more fully below.

The following example classification and regression models in planning and assessing experiments to determine formulations and solid forms illustrate some of the ways in which each type of model may be used. A classification model comprising a qualitative solubility assay may, for example, be used in conjunction with the FAST automated experimentation apparatus to assign a soluble/not soluble label to each individual experimental result set. A regression model comprising a quantitative solubility assay may, for example, be used with FAST to assign an estimated solubility, expressed for example in mg/ml. In conjunction with the CRYSTALMAX automated experimentation apparatus, a classification model may, for example, be used to assign a polymorph label to each individual experimental result set producing a solid form. A regression model may be used with CRYSTALMAX to, for example, provide an estimated nucleation time. For each model, the input may comprise experimental parameters and/or results.

Regression models may include (but are not limited to) linear regression, stepwise linear regression, additive models (AM), projection pursuit regression (PPR), recursive partitioning regression (RPR), alternating conditional expectations (ACE), additivity and variance stabilization (AVAS), locally weighted regression (LOESS), neural networks, Multivariate Adaptive Regression Splines (MARS), principal components regression, partial least squares regression, and support vector regression. Many other regression methods may be found in the literature, including Duda, Richard O., et al. Pattern Classification, second edition. John Wiley & Sons, Inc. 2001, which is incorporated herein by reference.

Classification models may include (but are not limited to) decision trees (generated by algorithm like C4.5, C5.0, or CART), support vector machines, neural networks, k-nearest neighbor classifiers, Bayesian classifiers (with probability density functions preferably determined using Gaussian Mixture Models or Parzen windowing), self-organizing maps. These and other classification models are described in Duda, Richard O., et al. Pattern Classification, second edition. John Wiley & Sons, Inc. 2001.

One or more models may preferably be generated based on the results of unsupervised learning and/or clustering applied to one or more collections of experimental result sets. In one preferred embodiment, described more fully below, a collection of individual experimental result sets is received, a similarity measure is calculated between a plurality of pairs of individual experimental result sets, and based on the similarity measure, a plurality of clusters of experimental result sets is determined, and one or more properties is determined for at least one solid form from each of at least two of the clusters. A three-dimensional visualization is preferably used to display the clusters. Preferably, each experimental result set in each cluster corresponds to a single solid form, preferably a single crystal polymorph. By characterizing the solid form corresponding to each cluster, solid form labels may be determined for each experimental result set for each cluster. Based on these labels and the experimental result sets and experimental parameters, a classifier model and/or a regression model may generated.

Unsupervised learning and clustering methods may include hierarchical clustering, including agglomerative and stepwise-optimal hierarchical clustering, k-means clustering, Gaussian mixture model clustering, or self-organizing-map (SOM)-based clustering, clustering using the Chameleon, DBScan, CURE, or Rock clustering algorithms, unsupervised Bayesian learning, Principal Component Analysis, Nonlinear Component Analysis, Independent Component Analysis, and multidimensional scaling. See Kohonen, T., “Self-organizing Maps”, Springer Series in Information Sciences, Vol. 30, Springer, Berlin, Heidelberg, New York, 3rd Extended Edition (2001); Duda, R., Hart, P., and Stork, D., “Pattern Classification”, John Wiley & Sons, 2^{nd} Edition (November 2000); Kaufinan, L., Rowseeaww, “Finding Groups in Data”, John Wiley & Sons, (1990); and Karypis G., et al., Chameleon: Hierarchical Clustering Using Dynamic Modeling, IEEE Computer, vol. 32, no. 8, at 68 (August 1999) (also referencing DBScan, CURE and Rock).

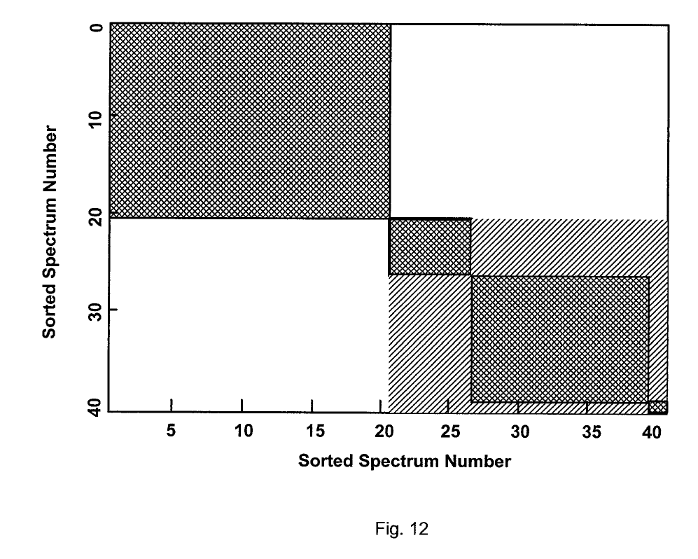

In the preferred method described below, the experimental result sets comprise Raman spectra, the similarity measure comprises the Tanimoto distance between bit-vectors representing peaks in Raman spectra, and the clustering method comprises hierarchical k-means clustering. The results of the preferred hierarchical clustering of Raman spectra described above are preferably displayed using a three-dimensional representation (two spatial coordinates plus color or shading) as shown in FIG. 12.

Based on the one or more generated models and/or multivariate visualizations, additional combinations of experimental parameters are determined to meet one or more experimental objectives. The experimental objectives preferably include determining boundaries between solid forms, determining regions in which desired properties of formulations change rapidly with respect to changes experimental parameters (not necessarily with respect to time), extrema (e.g. maxima or minima) of experimental results or parameters, regions within a class boundary, or regions of ambiguity or low confidence in classification or regression results.

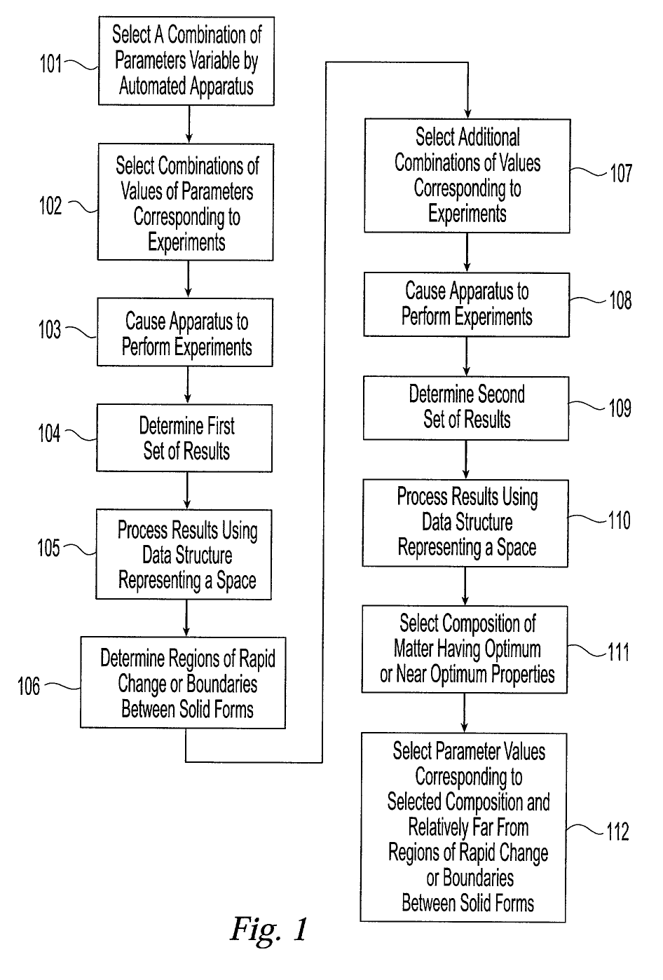

In one preferred embodiment, schematically illustrated in FIG. 1, data representing a collection of experimental results is processed as a collection of points in a space, such as a topological space, a metric space, or a vector space comprising dimensions corresponding to the dimensions of the experimental parameters 105. Based on this analysis, regions of the space are determined based on one or more experimental objectives such as determining boundaries between solid forms 106.

Based on this identification, the second (or subsequent) plurality of distinct combinations of values of the experimental parameters is preferably selected 107 to more fully define such boundaries or regions, and to include combinations of parameters as far as possible from such boundaries or regions. Alternatively, the second (or subsequent) plurality of distinct combinations of values of the experimental parameters my be selected 107 to more fully define regions in which desired properties of formulations change rapidly with respect to changes experimental parameters, extrema (e.g. maxima or minima) of experimental results or parameters, regions within a class boundary, and regions of ambiguity or low confidence in classification or regression results.

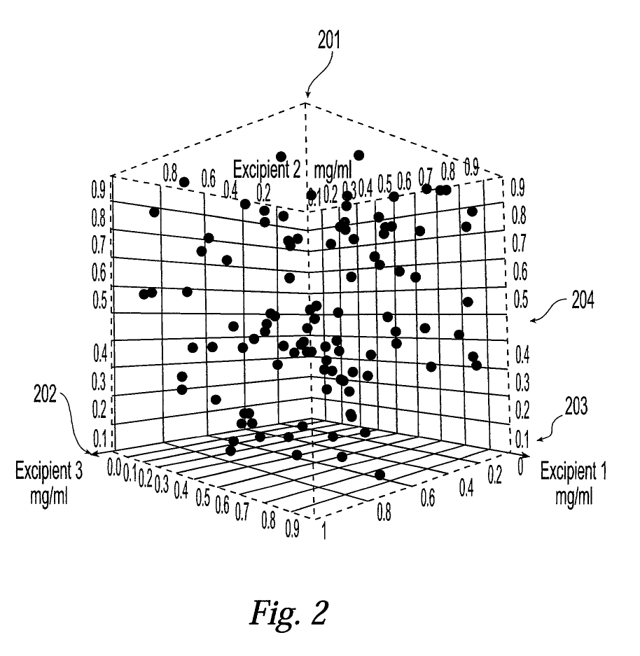

For example, the first collection of experimental results may preferably be processed by the computational informatics subsystem to display a multivariate visualization in which the experimental results are represented as points of varying size, color, shape or other indicia in a multidimensional space representing the space or a projection thereof, such as that shown in FIG. 2. By viewing such a visualization, an operator of the computational informatics subsystem may visually identify boundaries or regions of rapid change.

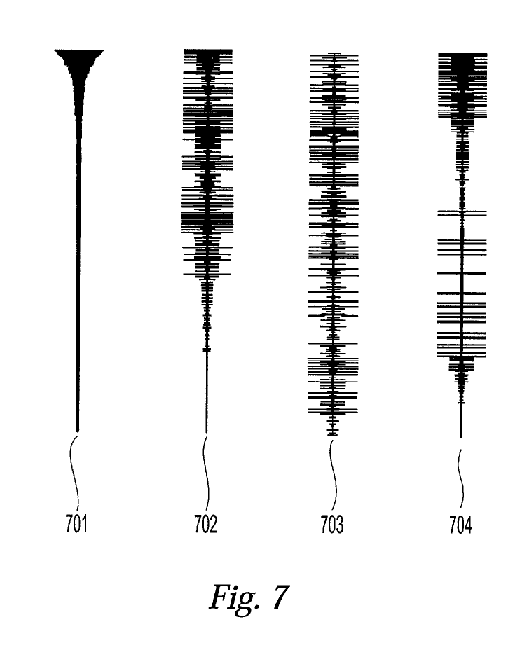

Alternatively, or in addition, other forms of multivariate visualization may be used, such as that depicted in FIG. 7. FIG. 7 depicts a multivariate visualization of one thousand experimental formulations, each with three excipients and one measured property of the formulation. Four axes 701, 702, 703 and 704 are depicted. Distinct formulations appear along the length of the axes, with each formulation appearing at the same place along all four axes. The width of each line on each axis is proportional to the normalized magnitude of the value represented by the axis for the corresponding experiment. For example, concentrations of excipients may be shown by the widths along axes 702, 703, and 704 and solubility may be shown by the widths along 701. A variety of multivariate visualization displays may be used. See, e.g. Tufte, E. R., The Visual Display of Quantitative Information (2d ed., Graphics Press 2001); Fayyad, U., et al., eds., Information Visualization in Data Mining and Knowledge Discovery (Morgan Kaufmann 2002) Alternatively, or in addition, combinations of values of the second plurality may be selected along a line or curve fitted to the data using a regression model, or selected based on a predicted classification. Other examples of selection include random or uniform selection within a range of values for results exhibiting desired properties, or selection within a range determined by use of one or more classification algorithms, such as a range classified as likely to correspond to a single solid form, or a range classified as likely to include a boundary between sets of experimental conditions within which two distinct solid forms are produced. Selection of additional values may also include a change of experimental parameters such as selection of different reagents or excipients likely to interact with observed species or solid forms. These and other preferred methods comprise other aspects of the invention, and are discussed in greater detail below.

Using the process informatics subsystem, the automated experimentation apparatus is activated to conduct a second set of experiments, each experiment of the second set corresponding to a distinct combination of values of the second plurality 108. The process informatics subsystem is also used to determine a second collection of experimental results of the second set of experiments, the second collection comprising a plurality of individual results, each individual result corresponding to a distinct experiment 109.

The computational informatics subsystem is then used to select a multicomponent chemical composition of matter or solid form based on the first collection of experimental results and the second collection of experimental results. Alternatively, additional iterations of experimentation may be performed prior to selecting the multicomponent chemical composition or solid form.

In one preferred embodiment, data representing the second or subsequent collection of experimental results is processed as a collection of points in a space such as topological space, metric space, or vector space comprising dimensions corresponding to the dimensions of the experimental parameters 110. Based on this processing, a set of experimental parameter values and a resulting multicomponent chemical composition of matter or solid form is preferably selected having optimum or near-optimum properties that do not change significantly within a region of the space corresponding to an expected range of conditions of manufacture, storage, and administration or use 111.

Planning and Assessing a Search for an Optimized Formulation

In one example preferred embodiment, an experimental search for a formulation having an optimized solubility is performed. This example is schematically illustrated in FIGS. 2-4. First, a combination of experimental parameters which may be varied by an automated experimentation apparatus is selected. In this example embodiment, the selected experimental parameters are concentrations of three selected excipients, schematically illustrated as a three-dimensional metric space in FIG. 2 comprising axes 201, 202, 203 of plot 204. A first plurality of distinct combinations of values of the experimental parameters is then determined, each combination corresponding to a distinct experiment. The combinations of values correspond to the coordinates of each of the data points 204 shown in FIG. 2.

Using the process informatics subsystem, the automated experimentation apparatus is caused to conduct a first set of experiments, each experiment of the first set corresponding to a distinct combination of the first plurality. In this example embodiment, each experiment comprises a sample formulation. Each sample formulation comprises one or more target active agents at fixed concentrations and a combination of excipients having concentrations corresponding to one of the data points 204 of FIG. 2. The process informatics subsystem is also used to determine a first collection of experimental results of the first set of experiments, the first collection comprising a plurality of individual result sets, where each individual result set corresponds to a distinct experiment. Each individual result set in this example embodiment comprises a measurement of the amount of an active component dissolved using standard technology such as mass spectroscopy, HPLC, UV spectroscopy, fluorescence spectroscopy, gas chromatography, optical density or colorimetry.

Using the process informatics subsystem, the measured experimental results are stored in a shared database, and thereby made available to the computational informatics subsystem. The computational informatics subsystem may then be used to visualize the experimental data in a high-dimensional multivariate display. In the display illustrated in FIG. 2, the size of plotted data points are used to depict the measured solubility of the active portion of the formulations corresponding to the data points 204, wherein larger sizes indicate greater solubility.

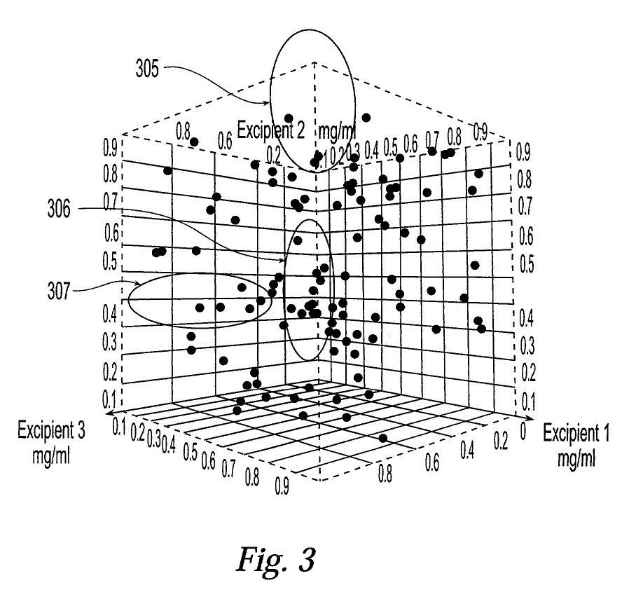

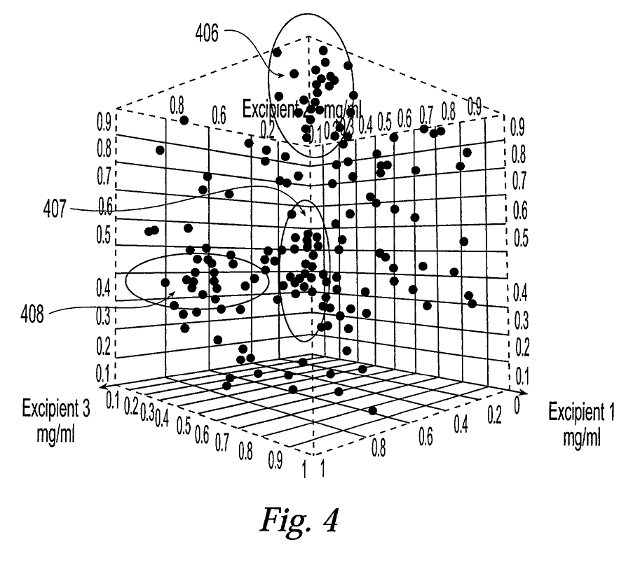

Using the computational informatics subsystem, a second plurality of distinct combinations of values of the experimental parameters is determined, based on the measured experimental results. For example, as shown in FIG. 3, certain experimental results or groups of experimental results 305, 306, 307 are identified as exhibiting measured results of interest. As shown in FIG. 4, additional data points 406, 407, 408 corresponding to distinct experiments may be selected to more accurately characterize the formulation near the results of interest.

In this example embodiment, a portion of the experimental results 305 of interest are solubility maxima or near-maxima in the sample. Another portion of the results of interest are groups of results 306 for which the rate of solubility change with respect to one or more experimental parameters is high relative to other groups of the sample. In this case, more experiments 406, 407 in this region will more accurately characterize the relationship between the experimental parameters and the change in solubility in the region. A third set of results of interest in this example are results 307 for which the rate of change of solubility with respect to one or more experimental parameters is low relative to other groups of the sample. In this situation, it is desirable to verify that the rate of change is low throughout the region by performing experiments 408 at a greater resolution to ensure that no changes in solubility have been missed by the resolution of the first set of experiments. Greater resolution is achieved by spacing the experiments 408 more densely in the region.

Using the process informatics subsystem, the automated experimentation apparatus is activated to conduct a second set of experiments, each experiment of the second set corresponding to a selected additional data point. In this example embodiment, each experiment comprises a sample formulation of the same one or more active agents and excipients as the first set of experiments. Alternatively, the concentration or identity of the one or more target active agents, or the identities of one or more excipients could be changed (or the numbers of excipients increased) for the second set of experiments. The process informatics subsystem is also used to determine a second collection of experimental results of the second set of experiments. In this example embodiment, the same measurement of solubility used for the first set of experiments is performed for the second set. Alternatively, a different measurement could be used for the second set.

Using the process informatics subsystem, measured experimental results are stored in the shared database, and thereby made available to the computational informatics subsystem. The computational informatics subsystem may then be used to visualize the experimental data in a multivariate display. In the display illustrated in FIG. 4, the size of plotted data points are used to depict the measured solubility of the active portion of the formulations corresponding to the data points of the second set of experiments 406. Additional iterations of selecting additional data points and automated experimentation may be performed.

Based on the collection of results, an optimum formulation is selected. In this solubility example, an optimum formulation is one having a high relative solubility, but comprising a combination of concentrations of excipients away from areas in which solubility changes relatively rapidly with concentration of one or more excipients. By avoiding a formulation in areas of rapid change, changes in the properties of the formulation due to expected variations of conditions of manufacture, storage, and administration or use are minimized.

Planning and Assessing a Massively Parallel Search for Solid Forms

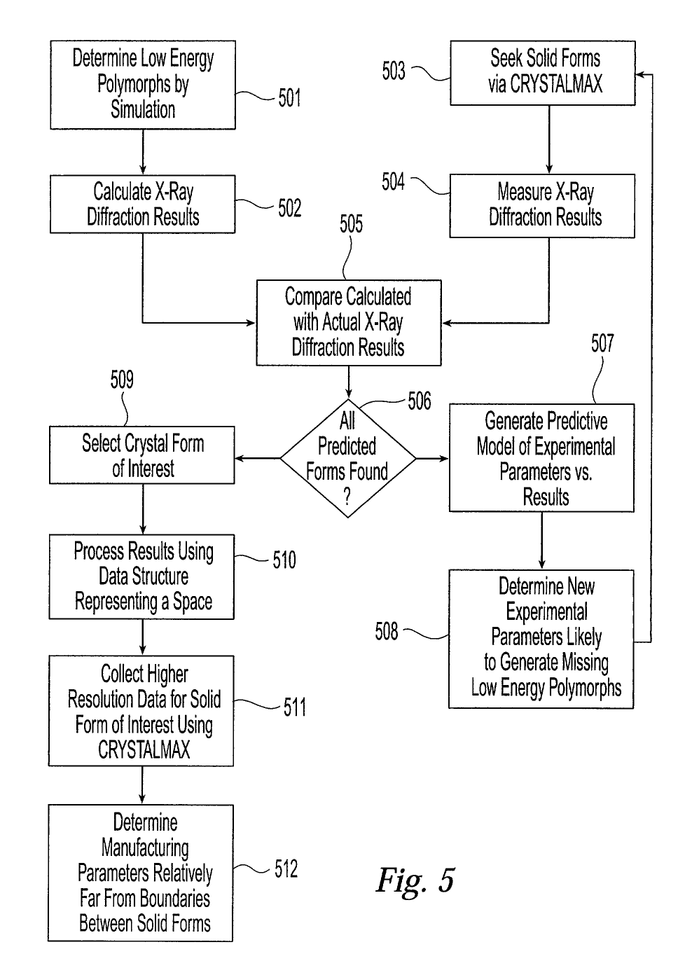

An example preferred method to assess a collection of experimental results in a search for novel or known solid forms is schematically illustrated in FIG. 5. The method comprises the steps of: determining low-energy crystal polymporphs via simulation model 501; characterizing the low-energy crystal polymorphs according to expected experimental results by standard techniques such as by calculated X-ray powder or single-crystal diffraction results 502; conducting a first collection of crystallization experiments 503; measuring a collection of actual experimental results such as actual X-ray powder diffraction for the crystals produced by the first collection of crystallization experiments 504; comparing the expected experimental results with the actual experimental results 505; determining if any lowest-energy structures were not included in the solid forms produced by a first collection of experiments 506.

Preferably, low-energy polymorphs are determined by using multivariate optimization such as hydrogen-bond-biased simulated annealing to locate a plurality of lowest-energy structures with the model.

One preferred energy function is crystal lattice energy, also referred to as the crystal binding or cohesive energy. Lattice energy is determined by summing all the pairwise atom-atom interactions between a central molecule and all the surrounding molecules. The calculation of lattice energy is discussed in Myerson, Molecular Modeling Applications in Crystallization, pp. 117-125, Cambridge University Press (1999), which is incorporated herein in its entirety by reference. The lattice energy is a useful parameter because its calculated value can be compared with the experimental enthalpy of sublimation. This allows one to verify the description of the intermolecular interactions by the force field in question.

An advantage of the calculated value of the crystal lattice energy is that it can be separated into specific interactions along certain directions and into the constituent atom-atom pair-wise contributions. This provides the link between molecular and crystal structures. The calculation of lattice energies thus provides a profile of the important intermolecular interactions that correspond to particular classes of compounds. It also provides an understanding of the nature of the intermolecular interactions that lead to a particular crystal packing arrangement.

Preferably, in performing atom-atom interactions, the potentials used include those that incorporate attractive or repulsive components, coulombic interaction, or hydrogen-bonding interaction. An example of these potentials include the Lennard-Jones potential, V_{vdw}=−A/r⁶+B/r¹², where A and B are the atom-atom parameters and r is the interatomic distance. The parameters A and B can be obtained by fitting the chosen potential to observable properties such as crystal structure, heats of sublimation, and hardness measurements. In accordance with the present invention, the results of a first principles calculation can also be used in the curve fitting step as an alternative to using actual experimental data to determine the parameters A and B. The coulombic interaction may be calculated using the equation V_{coul}=qᵢqⱼ/(Dr) where qᵢ and qⱼ are the charges on atoms i and j, D is the dielectric constant, and r is the interatomic distance. The hydrogen bonding potential may be calculated using a modified form of van der Waals potential such as a V_{vdw}=−A/r¹⁰ +B/r¹² potential instead of V_{vdw}=−A/r⁶+B/r¹² for the commonly used van der Waals potential function.

An example preferred multivariate optimization method used to search for a low energy crystal structure is the hydrogen-bond-biased simulated annealing monte carlo (SAMC) method described by Chin and co-workers in J. Am Chem. Soc. 1999, 121 2115-2122, the entirety of which is incorporated herein by reference. As described therein, one first builds and parameterizes a molecule using a molecular modeling program such as QUANTA, available from Molecular Simulations Inc., and then minimizes its energy using a program such as CHARMm, also available from Molecular Simulations Inc. (an academic version of the program, referred to as CHARMM, is also available from Harvard University). The molecular frame of reference is preferably positioned at the molecule's center of mass. Using preset limits of the unit cell and molecular rotation, a trial crystal structure with a given space group is built using a program such as CHARMM. Preferably, the limits used are: (a) a “loose” window for the lengths of the axes of the unit cell (for example, 30% greater than the largest molecular dimension as an upper limit and 3% less than the smallest dimension of the molecule as the lower limit); and (b) a range of angles corresponding to the allowable degree of molecular rotation.

Preferably, the above limits are chosen to ensure that any van der Waals interaction or contact present in the initially found crystal structure is not energetically unfavorable. In a preferred SAMC method and minimization procedure, the number of energetically favorable van der Waals interactions between the molecule and its crystalline environment increases with the lowering of the simulated annealing temperature.

To calculate the crystal energy (CE) of the trial crystal structure, CE can be expressed in terms of a Lennard-Jones type potential function and a coulombic interaction potential such as the one shown below.
CE=Εᵢⱼ[Aᵢⱼ/rᵢⱼ¹²−Bᵢⱼ/rᵢⱼ⁶+qᵢqⱼ/4πεᵢⱼ]
Thus, summing the contributions of the pairwise interactions between the ith atom on the initial molecule and the j^{th} atom of the surrounding molecules in the crystal allows calculation of CE. In the CE expression given above, rᵢⱼ is the distance between atoms i and j, Aᵢⱼ=(AᵢAⱼ)^{1/2} (for example) and Bᵢⱼ are the van der Waals parameters corresponding to atoms i and j, qᵢ represents the partial atomic charge of atom i, and εₒ is the permittivity of free space (8.854×10⁻¹² C²/Nm²).

In a preferred embodiment, unit cell dimensions are used as variables to be searched in the presence of the crystalline environment, and structures are chosen based on whether or not their hydrogen-bonding energies exceed a given value.

The SAMC method may be summarized as follows:
(1) building, parameterizing, and minimizing the energy of a molecule that will be used for the crystal construction.
(2) creating a reference crystal structure based on the molecule created in step (1) by randomly varying the unit cell parameters appropriate for the given crystal space group and the preselected molecular rotational constraint.
(3) calculating the crystal energy of the reference crystal and setting the value obtained as CEO.
(4) generating another crystal as in step (2) based on the given molecular constraints.
(5) minimizing the crystal energy using a gradient-descent method until the energy gradient falls below a certain limit.
(6) calculating the hydrogen-bonding energy of the energy-minimized crystal.
(7) rejecting the energy-minimized crystal if its hydrogen-bonding energy is greater than or equal to zero.
(8) denoting the crystal energy of the energy-minimized crystal as CE1 if its hydrogen-bonding energy is less than zero.
(9) comparing CE₁ with that of the previous crystal (CE₀ in the first iteration).
(10) setting CE₀=CE₁ if CE₁<CE₀.
(11) if CE₁>CE₀, calculating the Boltzmann weighting factor at a given temperature T, where the weighting factor is expressed by W=exp[−(CE₁−CE₀/k_{b}T)] where k_{b} is the Boltzmann constant and T is assigned an initial value (4,300° K., for example).
(12) generating a random number R between 0 and 1.
(13) comparing W in obtained in step (11) with the generated random number R in (12).
(14) rejecting the crystal structure corresponding to CE₁ if R≧W.
(15) setting CE₀=CE₁ if R<W.
(16) repeating steps 4-14 until a certain number of crystal structures have been obtained for a given temperature T.
(17) lowering the temperature by a certain value (500° K., for example), and repeating the entire procedure beginning with the last structure collected from the previous temperature.
(18) repeating steps 2-17 until the temperature has dropped below a given value.
(19) ranking all the structures collected at various temperatures from the lowest energy to the highest.
(20) selecting a plurality of the lowest energy structures from the ranking.

After selecting a plurality of the lowest energy structures from the ranking, the selected structures are characterized, according to expected experimental results, for solid forms corresponding to the structures. Preferably, the lowest energy structures are characterized by calculating X-ray powder diffraction results for each structure. Software for calculating X-ray powder diffraction results from a known structure, known as Cerius2, is available from Molecular Simulations Inc.

After characterizing the lowest energy structures according to expected experimental results, the process informatics subsystem preferably compares the expected experimental results with a set of actual experimental results from a first set of experiments. Based on the comparison, the process informatics subsystem assesses which, if any, of the lowest energy structures was produced by each experiment.

Preferably, the process informatics subsystem compares the expected experimental results with the actual experimental results of the first set of experiments by comparing calculated X-ray powder diffraction results for the lowest energy structures with experimentally measured X-ray powder diffraction results for the first set of experiments. The comparison is preferably performed by calculating a similarity measure of the expected experimental results and the actual experimentally measured results. Preferably, the similarity measure is calculated as
SI=d·F·d
where
d=s₁−s₂
is an n-vector that describes the difference between normalized sets of points in the calculated X-ray powder diffraction pattern s1 and the measured X-ray powder diffraction s₂.
And
F={F_{i,j}}
Where F is the n x n “fold matrix” described in Karfunkel, H. R.; Rohde, B.; Lousen, F. J. J.; Gdanitz, R.J.; Rihs, G. J. Comput. Chem. 1993, 14, 1125-1135, which is hereby incorporated in its entirety by reference.
Specifically,
F_{i,j}=1/(1+α(i−j)^{β})
where
the values of α and β are those empirically calibrated by Karfunkel: α=1.0×10⁻⁸ and β=4.

The similarity measure SI compares each point in one set with the set of nearby points in the other set, giving decreasing weight to points further away. Two identical sets would have an SI of zero. Larger values of SI imply greater dissimilarity between the calculated and measured spectra. This similarity measure was used by Chin and co-workers in the reference cited above and incorporated herein by reference.

Other forms of similarity measure may be used, however, it is preferable to use a measure that accounts for similarity over a neighborhood. Nevertheless, simpler methods such as the mean-square-difference between the two patterns may be used.

Based on the similarity measure, each experiment is classified as to which predicted lowest energy form was produced. This may be accomplished by classifying each experiment as the predicted low-energy structure having a calculated X-ray powder diffraction pattern most similar to the measured X-ray powder diffraction pattern according to the similarity measure applied. Using the preferred similarity measure, each experiment is classified as the predicted low-energy structure for which SI measure is the least. Preferably, a threshold is also applied, so that measured patterns for which the least SI is above the threshold are classified as “unknown.”

One preferred way of planning additional experiments to find missing expected solid forms is schematically illustrated in FIG. 5: generating a predictive model, such as a regression model, of the experimental parameters and results from the first set of experiments 507, and interpolating or extrapolating those results to determine sets of experimental parameters likely to produce predicted low-energy structures not produced in the first set of experiments 508.

One preferred method for generating a predictive model from the first set of experimental results is to apply Multivariate Adaptive Regression Splines (MARS) to the classified experimental results from the first set of experiments. MARS is described in J. H. Friedman, Multivariate Adaptive Regression Splines, SLAC PUB-4960 Rev, Tech Report 102 Rev (Stanford Linear Accelerator Center, 1990) at http://www.slac.stanford.edu/pubs/slacpubs/4750/slac-pub-4960.pdf which is incorporated herein by reference in its entirety. A computerized implementation of MARS is commercially available from Salford Systems of San Diego, Calif. (www.salford-systems.com). Other regression methods such as linear regression, stepwise linear regression, additive models (AM), projection pursuit regression (PPR), recursive partitioning regression (RPR), alternating conditional expectations (ACE), additivity and variance stabilization (AVAS), locally weighted regression (LOESS), neural networks, principal components regression, partial least squares regression, and support vector regression may also be used.

After generating a predictive model, the model is used to determine a second set of distinct combinations of experimental parameters that, according to the model, should produce predicted solid forms that were not produced in the first set of experiments. This may be accomplished by setting the response variable to a value corresponding to a missing predicted solid form and solving the predictive model for one or more sets of values of experimental parameters giving that result. For preferred predictive models, the solution may be found using algebraic or numerical methods readily apparent to those of ordinary skill in the art of using such predictive models.

Using the process informatics subsystem, the automated experimentation apparatus is activated to conduct a second set of experiments, each experiment of the second set corresponding to a distinct combination of experimental parameters determined using the predictive model. The second set of experimental results are preferably again compared against predicted experimental results as described above to classify the results according to predicted solid forms and to determine if all predicted low-energy structures have been produced.

Based on the collection of results, an optimum or near-optimum solid form is selected 509. Preferably, data representing the collection of experimental results is processed as a collection of points in a space, such as a topological space, metric space, or vector space comprising dimensions corresponding to the dimensions of the experimental parameters 510. Through such analysis, regions of the space in which the selected solid form is produced, and the boundaries between such regions and regions in which other forms or no solid forms are produced may be determined. Additional sets of experiments may be performed to define such regions with greater resolution 511. Preferably set of experimental parameters is thereby determined as far as possible from such boundaries 512. Such a set of parameters is advantageous for manufacture because small variations in manufacturing conditions are less likely to produce a solid form other than the selected form.

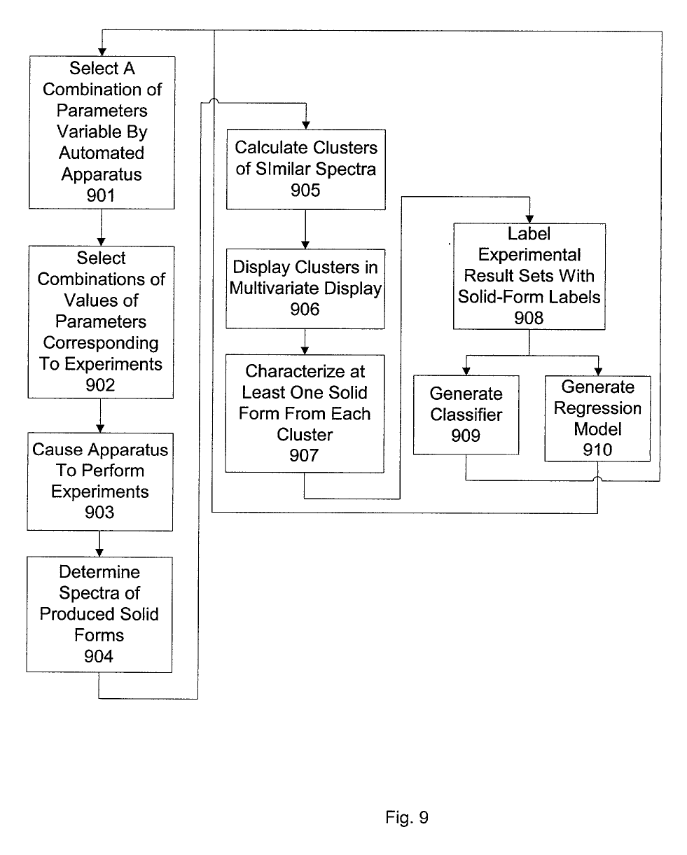

Another, more preferred, example method to assess collection of experimental results in a search for novel or known solid forms is schematically illustrated in FIG. 9. The method comprises the steps of: calculating a plurality of clusters of experiments resulting in a solid form based on a measure of similarity of characteristics of the experimental results and/or parameters 905; further characterizing at least one sample solid form from each cluster 907; based on the characterization, assigning a solid-form label to each experiment of each cluster 908. The method also comprises additional optional steps of: displaying clusters in a multivariate display 906, generating a classifier to assign a solid form label to an input comprising experimental parameters and/or results 909, generating a regression model 910 to estimate one or more expected property based on an input comprising experimental parameters and/or results, selecting a combination of experimental parameters variable by an automated experimentation apparatus 901, generating a plurality of sets of values of the experimental parameters, providing one or more of the sets to a classifier and/or regression model as input, and based on the output of the classifier and/or regression model, selecting combinations a plurality of sets of values of experimental parameters corresponding to experiments to be performed 902, providing selected sets of values of experimental parameters to an automated experimentation apparatus 903, and determining Raman spectra for experiments that produce solid forms 904. The method further optionally also comprises the step of: providing one or more individual experimental result sets as input to a classifier and/or regression model. The foregoing steps may be iterated an arbitrary number of times, with variations in the steps performed in each iteration. A preferred embodiment for implementing this method comprises the CRYSTALMAX automated experimentation apparatus configured to determine Raman spectra of solid forms, as described more fully in application No. 60/318,138, which is incorporated herein by reference.

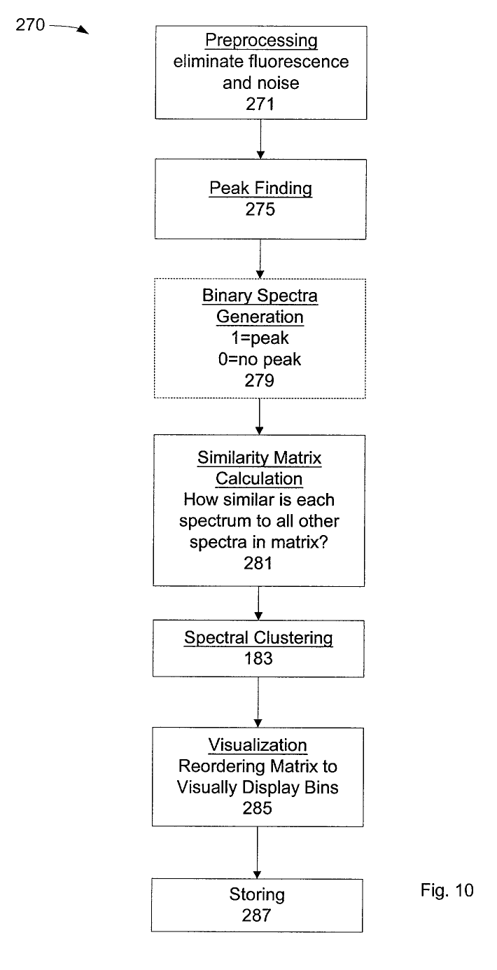

In one preferred embodiment, the computational informatics subsystem receives from the process informatics subsystem a plurality of Raman spectra, each spectrum corresponding to a distinct experiment. The computational informatics subsystem then preferably processes the spectra in six stages as schematically illustrated in the flow chart 270 in FIG. 10: preprocessing 271, peak finding 275, similarity matrix calculation 281, spectral clustering 283, and visualization 285. This process preferably also includes a binary spectra generation stage 279 between peak finding 275 and similarity matrix calculation 281. Each of these stages will be described in detail in the following sections. The following discussion relates to Raman spectra, but the same steps can easily be modified and applied to other types of spectra, or other forms of data.

Preprocessing

The purpose of the preprocessing step is to eliminate artifacts of the Raman spectra that are not caused by Raman scattering and to make the Raman scattering peaks as sharp as possible. Raman spectra often contain large fluorescence peaks spread over a broad spectral range and much smaller, narrower peaks caused by measurement, glass background, and instrument noise. Several different filtering techniques can be used in order to eliminate these deleterious features: Fourier filtering, wavelet filtering, matched filtering, etc. The preferred embodiment uses a matched filter approach where the filter kernel is a zero-mean, symmetric product of sinusoids matched approximately to an average Raman peak width. The specific form of the matched filter is given by the following equation:

$$k [ n ] = sin ( \frac{3 π n}{N - 1} ) \cdot sin ( \frac{π n}{N - 1} )$$

where N is the length of the kernel. In a more preferred embodiment, the matched filter equation includes a normalization term as follows:

$$k [ n ] = - \sqrt{\frac{4}{N - 1}} sin ( \frac{3 π n}{N - 1} ) \cdot sin ( \frac{π n}{N - 1} )$$

The normalization factor ensures that the magnitude of a filtered signal is about the same as the magnitude of the original, and that all peaks point in the right direction. In one embodiment, filtered points having a value less than zero are automatically set to equal zero.

Preferably, the bandwidth of the main kernel peak is set to be equal to or slightly smaller than the bandwidth of an average Raman peak. When matched filters of this type are viewed in the Fourier domain, they may be seen to perform as bandpass filters, almost completely attenuating low- and high-frequency spectral components. Furthermore, with the bandwidth of the filter kernel chosen to be equal to or slightly smaller than the average Raman peak bandwidth, this filter detects peaks that are very close to each other. A raw, unfiltered spectrum will often display two close peaks as a main peak with a “shoulder” on one of its sides. After a matched filtering step, though, the shoulder will often be distinguished as a separate peak. This separation is useful for the peak picking procedure described below.

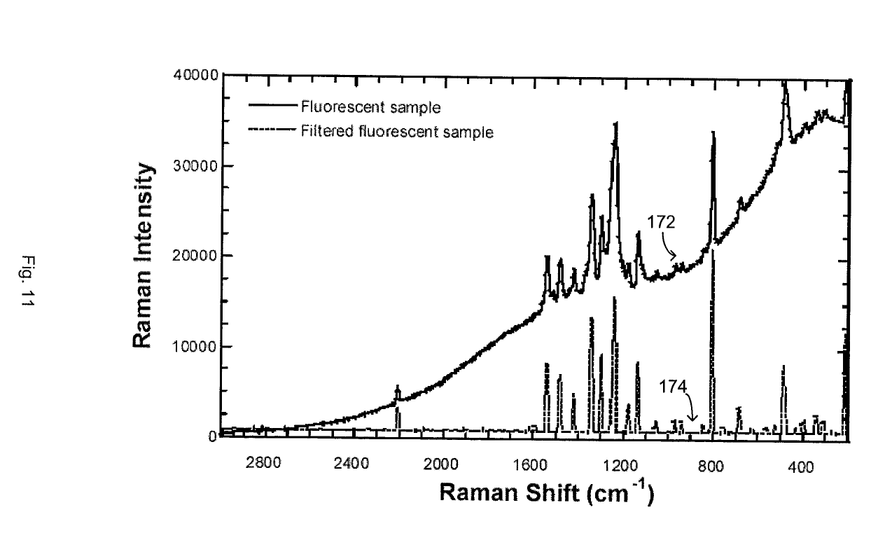

FIG. 11 shows a Raman intensity of a fluorescent sample as a function of Raman shift and the corresponding filtered spectra after the fluorescence has been removed.

Peak Finding

The process of finding peaks in a spectrum is an important aspect of many spectral processing techniques, and there are many commercially available programs for performing this task. Many variations of peak finding algorithms can be found in the literature. An example of a simple algorithm is to find the zero-crossings of the first derivative of a smoothed or unsmoothed spectrum, and then to select the concave down zero-crossings that meets certain height and separation criteria. For the preferred embodiment, the peak finding function available in the software provided with the Almega dispersive Raman spectrometer (Thermo Nicolet, OMNIC software) was used. This function allows the threshold and sensitivity values to be set by the user. The threshold sets the lowest peak height that will be counted as a peak, and the sensitivity controls how far apart each peak must be to count as a separate peak.

Binary Spectra Generation

Once the peaks have been found for all of the spectra, binary spectral representations are preferably created for all of the spectra. These binary spectra representations comprise vectors of ones and zeros. Each zero represents the absence of a peak feature and each one represents the presence of a peak feature. A peak feature is simply a peak that occurs within a certain spectral range, preferably a few wave numbers. The vectors for all of the spectra are preferably the same length and corresponding elements of these vectors correspond to the same peak feature.

In order to create these binary spectra, the peaks are clustered into ranges of peak features. The process used to perform this peak clustering is a modified form of a 1-dimensional iterative k-means clustering algorithm. The process begins with the picked peaks from a single spectrum. These peak positions are used to define the centers of peak feature ranges. The peak feature bins cover a range of wave numbers that can be specified by a user (the default is 5 wave numbers). The rest of the spectra are then iteratively added to the peak feature representation. At each step any peak that fits into a pre-existing peak feature range is added to that range. For any peak that does not fit into a range, a new range is created. Centers are not permitted to move so that peak feature ranges overlap. Then, the centers of all of the ranges are re-calculated and the peak feature ranges are re-defined relative to the new centers. This process can leave some peaks outside of an existing peak feature range. In this case, a new range is created for these peaks. This process creates a matrix with each row of the matrix corresponding to a binary spectrum specified in terms of range to which its peaks correspond. An example of such a matrix for five spectra is given below in TABLE 1.

TABLE 1

| Peak Position | 270 | 350 | 390 | 430 | 510 |
|---|---|---|---|---|---|
| Spectrum 1 | 1 | 1 | 0 | 1 | 1 |
| Spectrum 2 | 1 | 0 | 0 | 1 | 1 |
| Spectrum 3 | 1 | 1 | 0 | 0 | 0 |
| Spectrum 4 | 0 | 1 | 1 | 1 | 0 |
| Spectrum 5 | 1 | 1 | 0 | 1 | 1 |

In this matrix, Spectrum 1, for example, has a peak in each of the ranges corresponding to wave numbers 270, 350, 430 and 510, but does not have a peak in the bin associated with wave number 390.

Similarity Matrix Calculation

From either the spectra themselves, floating point or integer vectors representing the spectra, or from binary spectra representations such as those generated using the process described above, a similarity measure between pairs of spectra is calculated. Preferably, the similarity measure is calculated between each distinct pair of spectra. This similarity measurement is used to determine one or more clusters of similar spectra. Example similarity measurements include metric distances such as Hamming, Lp, or Euclidean distance, or non-metric similarity indices such a the Tversky similarity index (or its derivatives such as the Tanimoto or Dice coefficients) or functions thereof.

For a spectra to spectra similarity matrix, the following notation is used:
Nₘₙ=number of peak values in a first spectrum within 5 cm−1 of a peak value in a second spectrum;
Nₘ=number of peak values in the first spectrum; and
Nₙ=number of peak values in the second spectrum.

Similarity can then be calculated using various methods, e.g.,

$$Tanimoto ( m , n ) = \frac{{N}_{mn}}{{N}_{m} + {N}_{n} - {N}_{mn}}$$

FIGS. 12A and 12B were generated using the foregoing method.

For similarity matrices based on binary spectra, the following notation is used:
a=number of 1's in a first spectrum that are zeros in a second spectrum
b=number of 1's in a second spectrum that are zeros in the first spectrum
c=number of 1's in the first spectrum that are ones in the second spectrum

The above mentioned values can be calculated in the following manner:
Hamming distance: d=a+b
Euclidean distance: d=\sqrt{a+b}

$$\text{Tversky index:} t = \frac{c}{α a + β b + c}$$

Notably, as indicated previously, some of these metrics are related. For instance, the Tanimoto coefficient is equal to the Tversky index with α and β equal to 1. The Dice coefficient is equal to the Tversky index with α and β equal to 0.5. In a preferred embodiment, 1—Tanimoto coefficient is used as the (dis)similarity measure. Additional metrics, including metrics based on other metrics, may be used in alternative embodiments of the invention.

As noted, the selected similarity measure is preferably calculated for each distinct pair of spectra. This calculation may be represented as a symmetric similarity matrix with each element (ij) of the matrix representing the distance or similarity between spectra i and j.

Spectral Clustering

Using the similarity measure calculated between spectra, a clustering algorithm is applied to determine one or more clusters of similar spectra. A variety of different clustering algorithms may be used.

Hierarchical clustering, including agglomerative and stepwise-optimal hierarchical clustering, k-means clustering, Gaussian mixture model clustering, or self-organizing-map (SOM) -based clustering, clustering using the Chameleon, DBScan, CURE, or Rock clustering algorithms are some of the clustering methods that may be used. See Kohonen, T., “Self-organizing Maps”, Springer Series in Information Sciences, Vol. 30, Springer, Berlin, Heidelberg, New York, 3rd Extended Edition (2001); Duda, R., Hart, P., and Stork, D., “Pattern Classification”, John Wiley & Sons, 2^{nd} Edition (November 2000); Kaufman, L., Rowseeaww, “Finding Groups in Data”, John Wiley & Sons, (1990); and Karypis G., et al., Chameleon: Hierarchical Clustering Using Dynamic Modeling, IEEE Computer, vol. 32, no. 8, at 68 (August 1999) (also referencing DBScan, CURE and Rock).

In a preferred embodiment, hierarchical clustering is used as a first-pass method of spectral data processing. Using the information from the hierarchical clustering run, a step of k-means clustering is then performed with user-defined cluster numbers and initial centroid positions.

In another embodiment, the number of clusters can be automatically selected in order to minimize some metric, such as the sum-of-squared error or the trace or determinant of the within cluster scatter matrix. See Duda, R., Hart, P., and Stork, D., “Pattern Classification”, John Wiley & Sons, 2nd Edition (November 2000), which is incorporated herein by reference.

Visualization

Hierarchical clustering produces a dendrogram-sorted list of spectra, so that similar spectra are very close to each other. This dendrogram-sorted list is used to rearrange both axes of the original similarity matrix and then present the “sorted similarity” matrix in a coded manner wherein similarity indicia are used for each similarity region, including without limitation different symbols (such as cross-hatching), shades of color, or different colors. In a preferred embodiment, the “sorted similarity” matrix is presented in a color-coded manner, with regions of high similarity in warm colors and regions of low similarity in cool colors. Using this preferred three-dimensional (two spatial dimensions plus color) visualization, many clusters become apparent as warm-colored square regions of similarity along the matrix diagonal. These square regions represent a high degree of similarity between all of the spectral (i,j) pairs in those regions. An example preferred dendrogram-sorted similarity matrix with cross-hatching representing similarity is illustrated in FIG. 12.

It should be noted that the failure of the similarity matrix to present a diagonal form is to be expected with some types of samples, although the matrix is still useful in representing more complex similarity relationships. Furthermore, in some cases there can be similarity regions along more than one possible diagonal that correspond to different rearrangements. Such rearrangements result in off-diagonal similarity square regions becoming part of the diagonal similarity square regions.

Along with the matrix representation of the cluster data, it is also useful to show where all of the spectra and the cluster boundaries lie in a dimensionally reduced space (usually 2-dimensions). There are several ways to perform this dimensionality reduction. In a preferred embodiment, a linear projection is made of a binary spectra matrix onto its first two principal components. Alternatively, the chosen similarity matrix could be used in order to create a map of the data using multidimensional scaling.

An example Raman clustering application is written in Visual Basic (VB). This VB program allows a user to select a group of spectra and set processing parameters. Preprocessing is performed within the VB application and then the filtered spectra are sent to OMNIC for peak finding through the Macros/Pro DDE communication layer provided by OMNIC. Once peaks are found, binary spectrum and distance matrix generation is performed in the main VB application. Then, the distance matrix is sent to MATLAB through a socket communication layer. In MATLAB, clusters are generated and visualizations are created. These visualizations are made available to the main VB application through a web server present on the same machine as the MATLAB instance. The resulting visualization allows for the easy identification of groups of samples that all have similar physical structure.

After clusters have been calculated, it is desirable to correlate clusters with corresponding solid forms. This is preferably accomplished by selecting one sample, or preferably, a plurality of samples from each cluster and characterizing the selected sample or samples with additional experimental techniques, such as powder X-Ray diffraction and/or differential calorimetry. In a preferred embodiment, the clustering and techniques result in clusters of experimental results all of which produced the same solid form. Based on the additional experimental characterization, solid-form labels reflecting the solid form produced by the experiments of the cluster are associated with the experimental result sets by the computational informatics subsystem. These labels are preferably used in combination with the experimental result sets and the corresponding values of experimental parameters to generate one or more regression models and/or classifiers for use in planning and assessing further experiments, or estimating properties for conditions that have not been experimentally verified. For example, regression models may be used to estimate properties over a continuous range reflecting an infinite number of different conditions.

Generating a Maximally Diverse or Near-Maximally Diverse Set of Values of Experimental Parameters

One preferred approach to generating the first set of experiments in what may be a succession of iterative experiments is to systematically create a diverse set of experiments in a property/descriptor space of potential interest. Experimental parameters that may be varied by the automated experimentation apparatus must be selected, and values for those parameters determined, in order to conduct a set of experiments. Parameters may be selected by scientists acting on knowledge of the chemistry of the compound-of-interest, or the computational informatics system may guide the selection or suggest parameters by querying the database for similar compounds of interest and analyzing which descriptors were significant in prior experiments and/or simulations. The descriptors may then be mapped onto parameters that may be varied by the automated experimentation apparatus.

Many methods for solving the parameter selection problem in QSAR/QSPR are known. Three of the most popular solutions involve stepwise algorithms, genetic algorithms, and simulated annealing. These approaches may be adapted to parameter selection in the present system.

Stepwise algorithms are straightforward, but can lead to suboptimal results. A regression or classification is performed using each possible independent variable. The variable that performs the best is added to the model. The regression or classification is then performed again with the first variable and all possible second variables. The best second variable is then added to the model. Additional variables are added in similar fashion. This process is preferably continued a set number of times or until some measure of predictive ability reaches a minimum.

Genetic algorithms randomly create a population of models (with say 2-10 independent variables). Then regression or classification is performed with each of the models, and each model is scored for predictive power. Genetic operations (e.g. mutation—adding, deleting, or changing variables; or crosssover—taking variables from one model and mixing them with variables from another model) are then performed on the models based on their score. The process is iterated either a set number of times or until some condition is met (such as 10-100 iterations without improvement). The best model from any population is then selected. Simulated annealing starts with a randomly generated model (the “current solution”). The model is then perturbed (e.g. add, delete, or change a variable). The original model and the perturbed model are then scored for predictive ability (crossvalidated R² can be used for regression problems and correct classification percent can be used for classification problems). If the new model works better than the old one it becomes the new “current solution”. If the new model does not perform better than the old model, it can still become the new solution if the following condition is met:
A random number is generated uniformly between 0 and 1 and this random number is less than exp(−(R²_{current}−R²_{new})/T)
   The parameter T is a temperature parameter that is lowered over the course of iterations making it increasingly harder to accept a “worse” solution as the current solution This process is continues until a stopping condition is met (such as T reaching a predetermined value). See Zheng, W. and Tropsha, A, Novel Variable Selection Quantitative Structure-Property Relationship Approach Based on the k-Nearest-Neighbor Principle, J. Chem. Inf. Comput. Sci. 2000, 40, 185-194.

Another set of choices that a user or informatics system preferably makes are the manner in which the selected descriptors will be mathematically combined in order to create generate values for experimental parameters corresponding to experiments to be performed. For example, the arithmetic mean, the geometric mean, the standard deviation, or the geometric standard deviation may be computed by weight, by volume, or by mole fraction to combine descriptors mathematically to determine values for experimental parameters. The Hansen solubility parameter for a mixture, for example, is calculated by taking the arithmetic mean by volume of the Hansen solubility parameters for each of the mixture components.

The user or the informatics system preferably also chooses an algorithm for performing diversification and a metric by which diversification is to be measured. In one preferred embodiment, a tournament scheme is used in which for every mixture added to an experiment, 20-100 possible mixtures are randomly generated (random in their components), their mixture descriptors are calculated, and the one that is furthest from any other point already in the experiment is added to the experiment. This method seeks to maximize the minimum distance between any two experiments. Other algorithms may be used. The user also preferably selects the maximum number of components in an experiment.

Pharmaceutical Product Development “Pipeline” Management

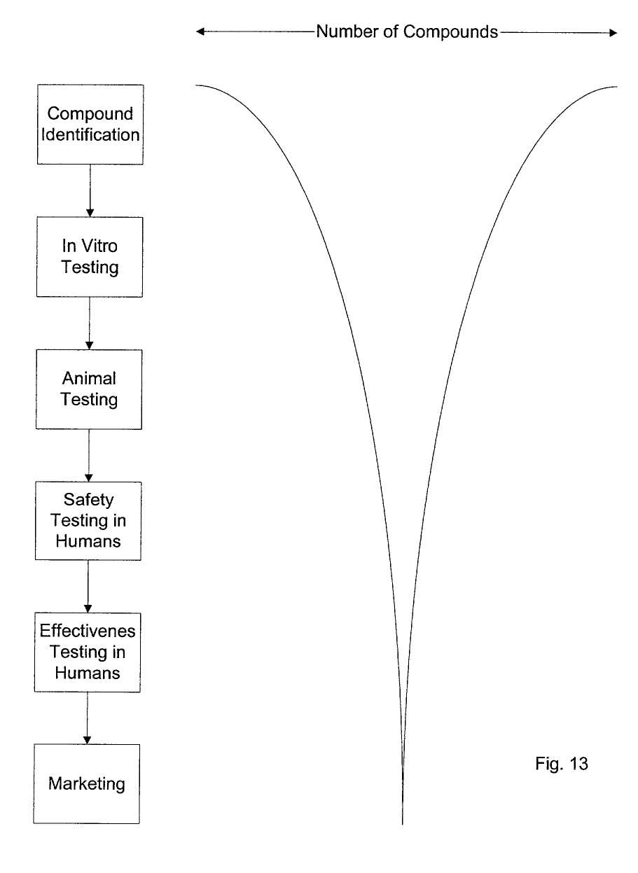

The methods and systems of the present invention may be used to great advantage in a system and method for pharmaceutical product development “pipeline” management. Pharmaceutical companies typically have a large number of compounds and new therapeutic uses of compounds at a variety of stages in the development, testing, and marketing process, or “pipeline.” Many of these stages, particular pharmaceutical testing stages, are dramatically expensive, and the number of compounds that proceed from one stage to the next is often reduced by an order of magnitude. As used herein, “pharmaceutical testing” means any investigations required or used for approval of a New Drug Application by the U.S. Food and Drug Administration. FIG. 13 schematically represents a simplified set of development, testing and marketing stages, and a corresponding qualitative indication of the manifold reduction in the number of compounds at each stage in the process.

The various stages in the product development process also provide data that change the ultimate form, formulation, manufacturing and distribution of the very small percentage of compounds in the development process that ultimately are marketed as pharmaceuticals. Many of these changes must be reflected in the Food and Drug Administration New Drug Application process, and may form a part of the labeling for the product. Because some results from very expensive portions of the product development process, such as human safety or effectiveness testing, may not be usable if the product must be reformulated or produced in a different solid form, it is desirable to determine the full range of solid forms of a candidate compound that may be produced, and to assess the properties of the selected solid form and formulation before large amounts of resources are expended on a solid form or formulation that differs from the final product.

Further, an important goal of market-driven pharmaceutical concerns is to maximize the profitability of the entire pipeline. By assessing the variety of solid forms of candidate compounds available, and at each stage of the process determining an optimum or near-optimum formulation that is consistent with the constraints on the candidate product imposed by other inputs in the development process such as safety and effectiveness, candidate compounds that pose expensive difficulties due to unfavorable solid forms or formulation difficulties may be lowered in priority until those difficulties are overcome, and those candidates with acceptable formulations and solid forms may be increased in priority.

The process of drug research and development is extremely complex and successfully taking a pharmaceutical product through the complex pathway from discovery of the API through subsequent safety and efficacy testing requires scientific expertise in many different areas. In the broadest view, the stages of drug research and development include, but are not limited to: discovery of an API; synthesis, and chemical and physical characterization of the API; pharmacology; pharmacokinetics; formulation development, synthesis, and chemical and physical characterization of formulations; animal safety testing; chemistry, manufacturing, and control testing; and clinical studies and human studies, including without limitation Phase I, Phase II, Phase III, and Phase IV, and various “sub-phases” of the same.

Most people are aware that the outcomes of animal and human studies are important to determine whether or not a drug candidate is approved for marketing; however, the research and testing relating to API and formulation synthesis, analysis, characterization, and stability required to determine and assure the identity, purity, quality, strength, physical characteristics and potency of the API(s), as well as formulated products containing the same, are the backbone of the research and development process, and occur throughout the life of a pharmaceutical product.

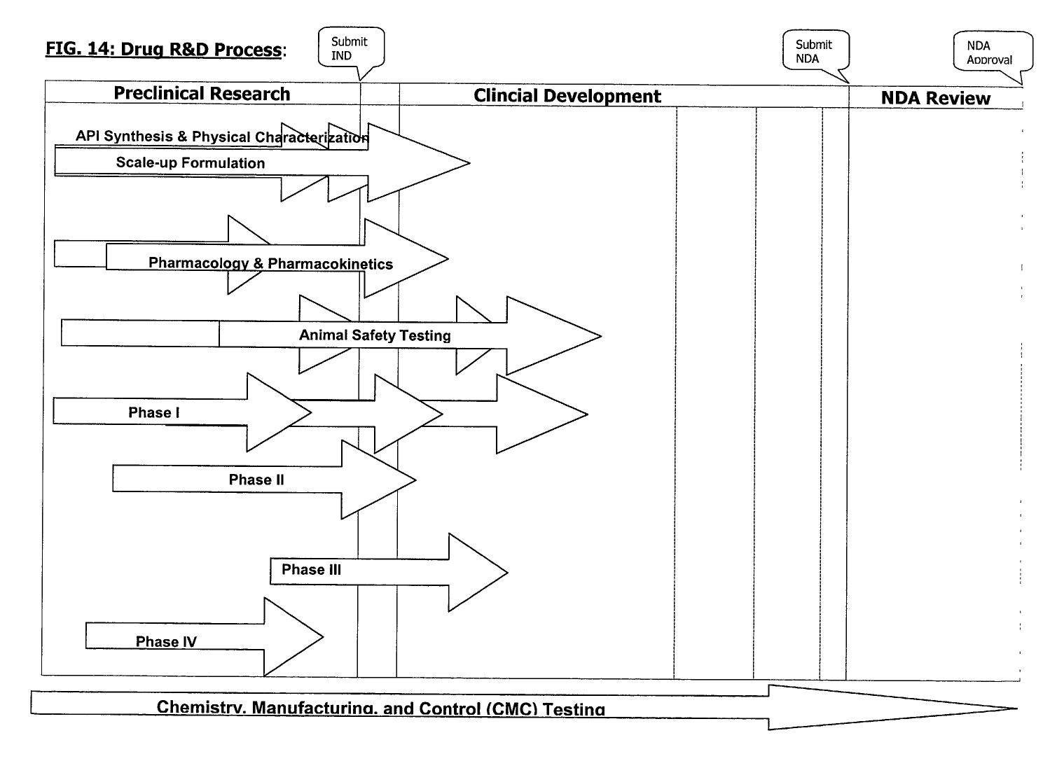

There are many ways to describe or group the pharmaceutical research and development process, and one example is shown in FIG. 14. It is important to recognize that while FIG. 14 is a linear representation, drug research and development is often a circular, or iterative process, as the results of one step may indicate that additional work in a previously carried out needs to be performed. Moreover, many steps occur throughout the research and development process, and beyond product launch. For example, chemistry, manufacturing and controls (CMC) testing (which includes, for example, analytical data, quality analysis, and stability testing in accordance with cGMP and other global regulatory standards) is essential at all phases of the research and development process, as well as the production and post-approval processes.

The terminology used herein is by way of example only and not meant to be limiting, since it is not uncommon in the pharmaceutical industry to see different terms used, or to change the designation of a particular step, although the goals of the steps are generally the same. For example, formulation development and scale-up (of API and formulation manufacturing processes) are sometimes designated as “preclinical” because of the nature of the work being performed, or they can be designated as “clinical” because they generally occur in association with and throughout the human testing phase of the process.

Another way the steps in the drug R&D process are sometimes grouped is to use the terms “nonclinical” and “clinical”, with nonclinical meaning anything that does not occur in or as a result of administration of a substance to humans, and clinical meaning anything that occurs in or as a result of administration of a substance to humans. Moreover, a number of steps occur in both groups or act as transition steps between preclinical and clinical (e.g., pharmacology and pharmacokinetics). Overall, the development of a drug or pharmaceutical is a stepwise process in which the goals of all phases or steps involve evaluating both the animal and human safety information relating to the compound-of-interest and a product containing the same.

A more detailed discussion of various steps of the drug research and development process is set forth below.

Preclinical Research

The goals of the preclinical or nonclinical safety evaluation include: characterization of toxic effects with respect to target organs, dose dependence, relationship to exposure, and potential reversibility. This information is important for estimating an initial safe starting dose for the human trials and the identification of parameters for clinical monitoring for potential adverse effects.

Preclinical research or testing includes, but is not limited to API synthesis, chemical and physical characterization of API, pharmacology, toxicology, metabolism, bioanalysis, pharmaceutical analysis, and biosafety testing. Preclinical research can also encompass studies that relate to the transition from preclinical to clinical, including Phase I studies, which typically provide a preliminary evaluation of a compounds safety, tolerance, and pharmacokinetics. In addition, preclinical research is carried out throughout all phases of the drug research and development process, and thorough preclinical research or testing maximizes the likelihood that a drug will be successful in the clinical phases of the process.

Chemical and physical characterization of a compound (or API) during the preclinical phase can include, but is not limited to: identification (e.g., by spectroscopic analysis); determining chromatographic purity; hygroscopocity determination; solubility studies; pKa determination; partitioning studies; characterization studies; short-term or accelerated stability; early formulation and excipient compatibility studies; developing chromatographic analysis of the API; residual solvents identification and quantitation; and reference standard certification.

Preclinical pharmacology and toxicology studies can include, but are not limited to, studies on or of: the pharmacological actions of the compound relating to its proposed therapeutic indication(s); defining the pharmacological properties of the compound; possible adverse effects of the compound; the toxicological effects of the compound relating to the compound's intended clinical uses, including without limitation assessing acute, sub-acute, and chronic toxicity (including single and repeated dose toxicity studies), and carcinogenicity; toxicities related to the compound's particular mode of administration or conditions of use; local tolerance; the effects of the compound on reproduction and on developing fetuses, or reproduction toxicity; genotoxicity; and the absorption, distribution, metabolism, and excretion of the compound in animals.

Metabolism studies relate to determining how a compound is absorbed, distributed, metabolized, and eliminated (ADME) from the body. In general, the early work on metabolism is carried out in in vitro studies, which are then followed by in vivo studies using relevant small and large animal models, and such studies particularly in small animals) can include whole-body autoradiography (WBA), which can provide qualitative &/or quantitative representations of a compound's distribution in the animal. In addition in vitro and animal studies, human metabolism (AME) can be studied in human clinical trials using C14 or another radiolabeled form of the compound.

Safety pharmacology studies in general are studies that investigate the potential undesirable pharmacodynamic effects of a compound on physiological functions when a subject is exposed to the compound in the proposed therapeutic dosage range and above. The goals of safety pharmacology studies include (1) identifying undesirable pharmacodynamic properties that may have relevance to human safety, (2) evaluating adverse pharmacodynamic and/or pathophysiological effects of a compound observed in toxicology and/or clinical studies, and (3) investigating the mechanism or mode of action of observed and/or suspected adverse pharmacodynamic effects.

Some safety pharmacology determinations or endpoints may be incorporated into the design of a various toxicology, pharmacokinetic, and clinical studies, while in other cases specific safety pharmacology studies are needed. The specific safety pharmacology studies that should be conducted and their design will vary based on the individual properties and intended uses of a compound or pharmaceuticals, but in general, factors to be considered when determining what safety pharmacology studies are needed include, but are not limited to: adverse effects related to the therapeutic class of the API, as the mechanism of action of the API may suggest certain adverse effects; adverse effects associated with members of the chemical or therapeutic class, but independent of the primary pharmacodynamic effects; ligand binding or enzyme assay data suggesting a potential for adverse effects; results from previous safety pharmacology studies, from secondary pharmacodynamic studies, from toxicology studies, or from human use that suggest further investigation to establish and characterize the relevance of the findings to potential adverse effects in humans.

In addition, a hierarchy of organ systems can used, wherein the hierarchy is established based on an organ's importance with respect to life-supporting functions. Vital organs or systems whose functions are acutely critical for life (e.g., the respiratory, cardiovascular, and central nervous systems), are the most important to assess in safety pharmacology studies. Other organ systems (e.g., the gastrointestinal or renal systems) whose functions can be transiently disrupted without causing irreversible harm, are of less immediate concern; however, safety pharmacology evaluation of these other systems may be of particular importance when considering factors such as likely clinical trial or patient populations (e.g., gastrointestinal tract in Crohn's disease, or immune system in immunocompromised patients.).

Preclinical or nonclinical studies such as pharmacokinetics and pharmacodynamics provide very important information for transitioning a compound from the preclinical stage to Phase I of the clinical stage. The areas or parameters that encompassed by pharmacokinetic studies include, without limitation, studies directed to: bioavailability/bioequivalence; absolute bioavailability; food effect; age or gender effects; nutritional effects; dose tolerance escalation; dose proportionality; controlled release; drug/drug interaction; and radiolabeled AME. Parameters that can be evaluated or measured in pharmacodynamic studies include, but are not limited to: gastric acid secretion; central nervous system (CNS) effects; cardiovascular effects; platelet aggregation; blood coagulation; bronchial challenge; wheal and flare response; endocrinology; and immunology

Clinical Development

Human clinical trials are conducted to demonstrate the efficacy and safety of a compound, beginning with a relatively low exposure in a small number of subjects, followed by clinical trials in which exposure usually increases by dose, duration, and/or size of the exposed patient population. Clinical trials are extended based on the demonstration of adequate safety in the previous clinical trial(s) as well as additional preclinical or nonclinical safety information that is available as the clinical trials proceed. Serious adverse clinical or nonclinical findings may influence whether clinical trials are continued and/or suggest the need for additional nonclinical studies and a reevaluation of previous clinical adverse events to resolve the issue.

While various terminology exists to refer to the phases of clinical development, the ICH guidance groups the phases by their purpose and objectives. Phase I clinical studies (also referred to as human pharmacology studies) are the first human exposure studies, and generally consist of single dose studies, followed by dose escalation and short-term repeated dose studies to evaluate pharmacokinetic parameters and tolerance. Phase I clinical trials are often conducted in healthy volunteers (or “normals”) but may also include patients suffering from the indication to be treated. Phase II clinical studies (also referred to as therapeutic exploratory studies) comprise exploratory efficacy and safety studies in patients Phase III clinical studies (also referred to as therapeutic confirmatory studies) comprise confirmatory clinical trials for efficacy and safety in patient populations.

Additional clinical studies performed after a compound or drug is approved that are related to the approved indication(s) are Phase IV studies (also referred to as therapeutic use studies or periapproval studies. Phase IV studies generally go beyond the prior demonstration of the drug's safety, effectiveness, and dose definition. While Phase IV studies are not necessary for product approval, they often are very important for optimizing the product's or drug's use, and such studies include, but are not limited to: additional drug-drug interaction studies; dose-response, or safety studies; and studies designed to support an extended claim under the approved indication (e.g., mortality/morbidity studies).

In some instances, Phase IV is also used to refer to studies focused on other aspects, including without limitation: expanding scientific understanding of the product; competitive comparisons to support marketing claims; safety confirmation, such as evaluating rare or infrequent adverse events; evaluating the drug for specialized markets such as pediatric use; and expansion of the product labeling and optimization of dose.

CMC Testing:

CMC Testing is continuous element of the pharmaceutical research and development process, and studies or tests that are referred to as CMC testing are often also grouped into other steps of the R&D process. Various CMC studies or tests include without limitation:

Discovery/preclinical to Phase I studies, including but not limited to, identification (e.g., by spectroscopic analysis), determining chromatographic purity, hygroscopocity determination, solubility studies, pKa determination, partitioning studies, characterization studies, short-term or accelerated stability, excipient compatibility studies, developing chromatographic analysis of the API, residual solvents identification and quantitation, and reference standard certification;

Phase I, II, and III studies, including but not limited to, stability testing of product used in Phase I studies, validation of analytical methods for API and Phase I product, release of Phase I, II and III clinical test materials and product for long-term toxicology studies, long term stability, refinements and validations of analytical methods resulting from product and process improvements, microbial testing, stress testing, extractability/leaching studies on product container & closures, cleaning validation support, and analysis of manufacturing process validation samples; and

Post-Approval studies, including but not limited to, quality control (QC) release of excipients, QC release of API, QC release of formulated product, scale-up and post-approval changes, and post-approval stability studies.

Preferred Embodiments of Process Informatics and Computational Informatics Subsystems

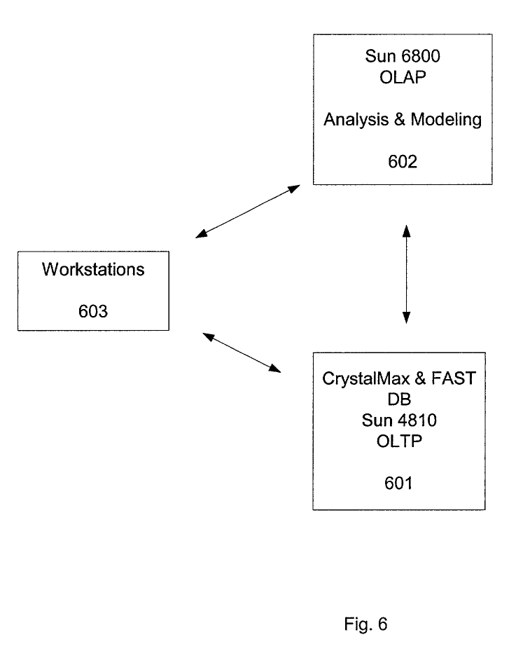

The architecture of a preferred example embodiment is schematically illustrated in FIG. 6. The computational informatics subsystem comprises two database systems, an Online Transaction Processing (OLTP) database system 601 and an Online Analytical Processing database system (OLAP) 602. The OLTP system 601 comprises an Oracle 8i object-oriented relational database management system with partitioning option running under Solaris 2.8 on a Sun Microsystems Sunfire 4810 with four 750 megahertz UltraSparc III CPUs and 4 gigabytes of RAM The OLAP system 602 comprises an Oracle 9i Data Warehouse database utilizing a snowflake schema running under Solaris 2.8 on a Sun Microsystems Sunfire 6800 with sixteen 900 megahertz UltraSparc III CPUs and 24 gigabytes of RAM. These systems are connected to 5 terabytes of disk space utilizing a Hitachi Thunder 9200 Storage Area Network. The connection is made between hosts and storage using redundant Brocade Silkworm 2800 fabric switches and 100 megabyte fiber optic lines. The FAST and Crystalmax platforms preferably share common informatics subsystems. The informatics for all of the various flavors of platform development are preferably consolidated into one application and database instance, being run on the systems described above. Windows systems 603 preferably comprise a variety of personal workstation hardware ranging from typical desktop PCs to high-performance workstations with visualization hardware.

The OLTP 601 and OLAP 602 are preferably interconnected with gigabit Ethernet. Windows systems 603 are typically connected to the computational informatics subsystem by a variety of heterogeneous networks, including the Internet.

The foregoing has demonstrated the pertinent and important features of the present invention. One of skill in the art will be appreciate that numerous modifications and embodiments may be devised. Therefore, it is intended that the appended claims cover all such modifications and embodiments.

1. A method for determining a formulation of a pharmaceutical, comprising the steps of:
performing high-throughput formulation screening of the pharmaceutical;
computing an optimization algorithm to select a plurality of molecular descriptors and a model accepting the molecular descriptors as parameters to optimize the predictive power of the model;
determining the formulation of the pharmaceutical.

2. A method for generating a plurality of solid forms of a pharmaceutical, comprising the steps of:
performing high-throughput solid-form screening of the pharmaceutical;
computing an optimization algorithm to select a plurality of molecular descriptors and a model accepting the molecular descriptors as parameters to optimize the predictive power of the model;
determining the formulation of the pharmaceutical.

3. The method of claim 1, further comprising the steps of:
generating values of experimental parameters using the model;
performing high-throughput screening using the generated values.
comparing the high-throughput experimental results with the results predicted by the model;
adjusting the model based on the high-throughput experimental results.

4. The method of claim 2, further comprising the steps of:
generating values of experimental parameters using the model;
performing high-throughput screening using the generated values.
comparing the high-throughput experimental results with the results predicted by the model;
adjusting the model based on the high-throughput experimental results.

5. The method of claim 3 or 4, wherein the generated values are targeted to find an extremum of an expected property of an experiment.

6. The method of claim 3 or 4, wherein the generated values are targeted to determine boundaries between solid forms.

7. The method of claim 3 or 4, wherein the generated values are targeted to determine regions in which desired properties of formulations change rapidly with respect to changes experimental parameters.

8. The method of claim 3 or 4, wherein the generated values are targeted to determine regions in which desired properties of formulations change slowly with respect to changes experimental parameters.

9. The method of claim 3 or 4, wherein the generated values are targeted to a region of ambiguity or low confidence in classification or regression results.

10. The method of claim 1, 2, 3 or 4, wherein the predictive power is determined with respect to an extremum of an expected property of an experiment.

11. The method of claim 2, wherein the predictive power is determined with respect to boundaries between solid forms.

12. The method of claim 1, 2, 3 or 4, wherein the predictive power is determined with respect to regions in which desired properties of formulations or solid forms change rapidly with respect to changes in experimental parameters.

13. The method of claim 1, 2, 3 or 4, wherein the predictive power is determined with respect to one or more regions within class boundaries.

14. The method of claim 1, 2, 3 or 4, wherein the optimization algorithm comprises a stepwise algorithm.

15. The method of claim 1, 2, 3 or 4, wherein the optimization algorithm comprises a genetic algorithm.

16. The method of claim 1, 2, 3 or 4, wherein the optimization algorithm comprises simulated annealing.

17. The method of claim 1, 2, 3 or 4, wherein the model is a regression model.

18. The method of claim 1, 2, 3 or 4, wherein the model is a classifier.

19. The method of claim 1, 2, 3 or 4, wherein the model comprises linear regression.

20. The method of claim 1, 2, 3 or 4, wherein the model comprises stepwise linear regression.

21. The method of claim 1, 2, 3 or 4, wherein the model comprises an additive model.

22. The method of claim 1, 2, 3 or 4, wherein the model comprises projection pursuit regression.

23. The method of claim 1, 2, 3 or 4, wherein the model comprises recursive partitioning regression.

24. The method of claim 1, 2, 3 or 4, wherein the model comprises alternating conditional expectations.

25. The method of claim 1, 2, 3 or 4, wherein the model comprises additivity and variance stabilization.

26. The method of claim 1, 2, 3 or 4, wherein the model comprises locally weighted regression.

27. The method of claim 1, 2, 3 or 4, wherein the model comprises a neural network.

28. The method of claim 1, 2, 3 or 4, wherein the model comprises multivariate adaptive regression splines.

29. The method of claim 1, 2, 3 or 4, wherein the model comprises principal components regression.

30. The method of claim 1, 2, 3 or 4, wherein the model comprises partial least squares regression.

31. The method of claim 1, 2, 3 or 4, wherein the model comprises support vector regression.

32. The method of claim 1, 2, 3 or 4, wherein the model comprises a decision tree.

33. The method of claim 32, wherein the decision tree is generated an algorithm selected from the set consisting of C4.5, C5.0 or CART.

34. The method of claim 1, 2, 3 or 4, wherein the model comprises a support vector machine.

35. The method of claim 1, 2, 3 or 4, wherein the model comprises a k-nearest neighbor classifier.

36. The method of claim 1, 2, 3 or 4, wherein the model comprises a bayesian classifier.

37. The method of claim 36, wherein the model further comprises a probability density function determined using a Gaussian Mixture Model.

38. The method of claim 36, wherein the model further comprises a probability density function determined using Parzen windowing.

39. The method of claim 1, 2, 3 or 4, wherein the model comprises a self-organizing map.

40. The method of claim 1, 2, 3 or 4, wherein an approximately maximally diverse set of values of experimental parameters for high-throughput screening is generated using a diversification algorithm and a metric for measuring diversification.

41. The method of claim 1, 2, 3 or 4, wherein a set of values of experimental parameters for high-throughput screening is generated based on a structure-activity model.

42. A method for selecting a compound for further testing, comprising the steps of:
receiving information of a plurality of compounds;
performing high-throughput solid-form screening of at least one of the plurality of compounds to identify at least one solid-form;
based on the at least one property of each identified solid-form, selecting at least one of the plurality of compounds for further testing.

43. A method for selecting a compound for further testing, comprising the steps of:
receiving information of a plurality of compounds;
performing high-throughput formulation screening on at least one of the plurality of compounds;
based on at least one tested property, selecting at least one of the plurality of compounds for further testing.

44. A method for selecting a solid form of a compound for further testing, comprising the steps of:
receiving information of a compound;
performing high-throughput solid-form screening to identify at least two solid forms of the compound;
based on the results of the high-throughput solid-form screening, selecting a solid form of the compound for further testing.

45. A method for selecting a formulation of a compound for further testing, comprising the steps of:
receiving information of a compound;
performing high-throughput formulation screening of the compound;
based on the results of the high-throughput formulation screening, selecting a formulation of the compound for further testing.

46. A method for determining whether to further test at least one compound, comprising the steps of:
receiving information of the at least one compound;
performing high-throughput formulation screening of the at least one compound;
based on at least one tested property, determining whether to further test the at least one compound.

47. A method for determining whether to further test at least one compound, comprising the steps of:
receiving information of the at least one compound;
performing high-throughput solid-form screening of the at least one compound;
based on at least one tested property, determining whether to further test the at least one compound.

48. The method of claim 42, 43, 44, 45, 46, or 47, further comprising the steps of:
based on the results of the high-throughput screening, generating a model to estimate at least one property of the compound.

49. The method of claim 48, wherein the model is a regression model.

50. The method of claim 48, wherein the model is a classifier.

51. The method of claim 48, wherein the at least one property comprises solubility.

52. The method of claim 48, wherein the at least one property comprises bioavailability.

53. The method of claim 48, wherein the at least one property comprises dissolution.

54. The method of claim 53, wherein the at least one property further comprises dissolution time.

55. The method of claim 48, wherein the at least one property comprises stability.

56. The method of claim 48, wherein the at least one property comprises permeability.

57. The method of claim 48, wherein the at least one property comprises partitioning.

58. The method of claim 48, wherein the at least one property comprises a mechanical property.

59. The method of claim 58, wherein the mechanical property comprises compressibitility.

60. The method of claim 58, wherein the mechanical property comprises compactibility.

61. The method of claim 58, wherein the mechanical property comprises a flow characteristic.

62. The method of claim 58, wherein the mechanical property comprises compressibitility.

63. The method of claim 48, wherein the at least one property comprises color.

64. The method of claim 48, wherein the at least one property comprises taste.

65. The method of claim 48, wherein the at least one property comprises smell.

66. The method of claim 48, wherein the at least one property comprises absorption.

67. The method of claim 48, wherein the at least one property comprises toxicity.

68. The method of claim 48, wherein the at least one property comprises metabolic profile.

69. The method of claim 48, wherein the at least one property comprises potency.

70. The method of claim 1, 2, 3, or 4 further comprising the steps of:
based on the results of the high-throughput screening, generating a classifier to assign each solid form to a class.

71. The method of claim 70, wherein at least one class corresponds to a crystal polymorph.

72. The method of claim 70, wherein at least one class corresponds to a crystal habit.

73. The method of claim 70, wherein at least one class corresponds to a salt.

74. The method of claim 70, wherein at least one class corresponds to a hydrate.

75. The method of claim 70, wherein at least one class corresponds to a solvate.

76. The method of claim 70, wherein at least one class corresponds to a defined particle size range.

77. The method of claim 48, wherein the model comprises linear regression.

78. The method of claim 48, wherein the model comprises stepwise linear regression.

79. The method of claim 48, wherein the model comprises an additive model.

80. The method of claim 48, wherein the model comprises projection pursuit regression.

81. The method of claim 48, wherein the model comprises recursive partitioning regression.

82. The method of claim 48, wherein the model comprises alternating conditional expectations.

83. The method of claim 48, wherein the model comprises additivity and variance stabilization.

84. The method of claim 48, wherein the model comprises locally weighted regression.

85. The method of claim 48, wherein the model comprises a neural network.

86. The method of claim 48, wherein the model comprises multivariate adaptive regression splines.

87. The method of claim 48, wherein the model comprises principal components regression.

88. The method of claim 48, wherein the model comprises partial least squares regression.

89. The method of claim 48, wherein the model comprises support vector regression.

90. The method of claim 48, wherein the model comprises a decision tree.

91. The method of claim 48, wherein the decision tree is generated an algorithm selected from the set consisting of C4.5, C5.0 or CART.

92. The method of claim 48, wherein the model comprises a support vector machine.

93. The method of claim 48, wherein the model comprises a k-nearest neighbor classifier.

94. The method of claim 48, wherein the model comprises a bayesian classifier.

95. The method of claim 94, wherein the model further comprises a probability density function determined using a Gaussian Mixture Model.

96. The method of claim 94, wherein the model further comprises a probability density function determined using Parzen windowing.

97. The method of claim 48, wherein the model comprises a self-organizing map.

98. The method of claim 42, 43, 44, 45, 46, or 47 further comprising the steps of:
applying at least one unsupervised learning or clustering algorithm to at least a subset of the results of the high-throughput screening.

99. The method of claim 98 wherein the unsupervised learning or clustering algorithm comprises hierarchical clustering.

100. The method of claim 98 wherein the unsupervised learning or clustering algorithm comprises agglomerative hierarchical clustering.

101. The method of claim 98 wherein the unsupervised learning or clustering algorithm comprises stepwise-optimal hierarchical clustering.

102. The method of claim 98 wherein the unsupervised learning or clustering algorithm comprises k-means clustering.

103. The method of claim 98 wherein the unsupervised learning or clustering algorithm comprises gausssian mixture model clustering.

104. The method of claim 98 wherein the unsupervised learning or clustering algorithm comprises self-organizing map-based clustering.

105. The method of claim 98 wherein the unsupervised learning or clustering algorithm comprises clustering using the Chameleon, DBSCan, CURE or ROCK algorithms.

106. The method of claim 98 wherein the unsupervised learning or clustering algorithm comprises unsupervised Bayesian learning.

107. The method of claim 98 wherein the unsupervised learning or clustering algorithm comprises principal component analysis.

108. The method of claim 98 wherein the unsupervised learning or clustering algorithm comprises nonlinear component analysis.

109. The method of claim 98 wherein the unsupervised learning or clustering algorithm comprises independent component analysis.

110. The method of claim 98 wherein the unsupervised learning or clustering algorithm comprises multidimensional scaling.

111. A method for selecting a compound for priority testing, comprising the steps of:
receiving information of a plurality of compounds;
performing high-throughput solid-form screening of at least one of the plurality of compounds to identify at least one solid-form;
based on the at least one property of each identified solid-form, selecting at least one of the plurality of compounds for further testing.

112. A method for selecting a compound for priority testing, comprising the steps of:
receiving information of a plurality of compounds;
performing high-throughput formulation screening on at least one of the plurality of compounds;
based on at least one tested property, selecting at least one of the plurality of compounds for further testing.

113. A method for selecting a solid form of a compound for priority testing, comprising the steps of:
receiving information of a compound;
performing high-throughput solid-form screening to identify at least two solid forms of the compound;
based on the results of the high-throughput solid-form screening, selecting a solid form of the compound for further testing.

114. A method for selecting a formulation of a compound for priority testing, comprising the steps of:
receiving information of a compound;
performing high-throughput formulation screening of the compound;
based on the results of the high-throughput formulation screening, selecting a formulation of the compound for further testing.

115. A method for determining whether to priority test at least one compound, comprising the steps of:
receiving information of the at least one compound;
performing high-throughput formulation screening of the at least one compound;
based on at least one tested property, determining whether to further test the at least one compound.

116. A method for determining whether to priority test at least one compound, comprising the steps of:
receiving information of the at least one compound;
performing high-throughput solid-form screening of the at least one compound;
based on at least one tested property, determining whether to further test the at least one compound.

117. A method for selecting a solid form of a compound for further testing, comprising the steps of:
receiving information of a compound;
performing high-throughput formulation screening to identify at least two solid forms of the compound;
based on the results of the high-throughput formulation screening, selecting a solid form of the compound for further testing.
